(19) United States
(12) Patent Application Publication
(10) Pub. No.: US 2023/0290511 A1
(43) Pub. Date: Sep. 14, 2023

(54) TRAINING OF COMPUTERIZED MODEL AND DETECTION OF A LIFE-THREATENING CONDITION USING THE TRAINED COMPUTERIZED MODEL

(71) Applicant: DATAHAMMER OY, Tampere (FI)

(72) Inventor: Antti KALLONEN, Tampere (FI)

(21) Appl. No.: 18/248,272

(22) PCT Filed: Oct. 11, 2021
(86) PCT No.: PCT/FI2021/050674

§ 371 Date: Apr. 7, 2023

(30) Foreign Application Priority Data
Oct. 9, 2020 (FI) 20205993

(51) Int. Cl. G16H 50/20; G16H 50/30; G06N 3/045

(52) U.S. Cl. CPC G16H 50/20; G06N 3/045; G16H 50/30

(57) ABSTRACT

There is provided detection of one or more life-threatening conditions with the help of a computerized model. A method for training a computerized model comprises receiving time-domain sample sequences of measurements of at least two biosignals from subjects; determining on the basis of computer-readable data from a subject database, information indicating timing of one or more life-threatening conditions of subjects; windowing the received time-domain sample sequences on the basis of a predefined window length; generating two-dimensional power spectral densities, 2D PSDs, of the windowed time-domain sample sequences; labeling the generated 2D PSDs to indicate a relationship to a life-threatening condition on the basis of the determined information indicating timing of one or more life-threatening conditions of the subjects; training a computerized model for detection of a life-threatening condition on the basis of the labeled 2D PSDs.

### CROSS REFERENCES

This application is a national phase application under 35 U.S.C. 371 of PCT/FI2021/050674, filed on Oct. 11, 2021 and claiming priority to Finnish national application FI 20205993, filed on Oct. 9, 2020.

### TECHNICAL FIELD

The examples and non-limiting embodiments relate generally to detection of a life-threatening condition based on and more particularly to computer-aided detection of a life-threatening condition based on measurements of biosignals.

### BACKGROUND

Most life-threatening conditions are characterized by disturbances in the homeostasis of multiple organ systems. Common life-threatening conditions such as sepsis, cardiac arrest and seizures can be identified from changes in biosignals measured from an organism. However, it has been difficult to identify specific signs for each life-threatening condition from the noisy signals recorded using simple numerical approaches. It is an unmet clinical need to be able to reliably identify life-threatening conditions as early as possible in order to take appropriate actions to prevent their progression and reduce associated mortality and morbidity. For example in the case of sepsis this could mean early antibiotic administration which would prevent the progression of the inflammatory state.

Measurement of biosignals from an organism, such as a human patient, can be performed invasively or non-invasively. Non-invasive sensors have multiple advantages over invasive sensors as they do not require invasive procedures performed on the patient by clinical experts. Therefore, when using non-invasive sensors, computer-aided detection of a life-threatening condition can be performed in low-resource settings, general wards, prehospital care or even at home of the patient as there is no need for specialized clinical staff and the infection risk is also minimized.

Electrocardiography is the process of producing an electrocardiogram (ECG). ECG is a graph of voltage versus time of the electrical activity of the heart using electrodes placed on the skin. These electrodes detect the small electrical changes that are a consequence of cardiac muscle depolarization followed by repolarization during each cardiac cycle (heartbeat). Changes in the normal ECG pattern occur in numerous cardiac abnormalities, including cardiac rhythm disturbances (such as atrial fibrillation and ventricular tachycardia), inadequate coronary artery blood flow (such as myocardial ischemia and myocardial infarction), and electrolyte disturbances (such as hypokalemia and hyperkalemia).

Sepsis may also be detected from ECG signal by an experienced medical professional using numerical approaches. Most common approach is to calculate Heart Rate Variability (HRV) which has been shown to have some predictive value in diagnosing sepsis journals.lww.com/md-journal/fulltext/2020/01240/depressed_sympathovagal_modulation_indicates.72.aspx. However, diagnosing sepsis using changes in ECG such as HRV is very difficult even for experienced clinical professionals as HRV features exhibit low sensitivity and specificity in diagnosing the condition.

ECG signal is usually also very noisy so that noise tolerant methods are required to extract meaningful information. Some typical noise sources to ECG signal are Electromyogram noise, Additive white Gaussian noise, and power line interference, for example.

Electroencephalography (EEG) is an electrophysiological monitoring method to record electrical activity of the brain. EEG measures voltage fluctuations resulting from ionic current within the neurons of the brain. EEG is most often used to diagnose epilepsy. It is also used to diagnose sleep disorders, depth of anesthesia, coma, encephalopathies, seizures and brain death. EEG signals or their spectral content is used in diagnosis of these conditions in a very controlled clinical environment. Specific EEG changes related to multiple life-threatening conditions are unknown.

A photoplethysmogram (PPG) is an optically obtained plethysmogram that can be used to detect blood volume changes in the microvascular bed of tissue. A PPG is often obtained by using a pulse oximeter which illuminates the skin and measures changes in light absorption. A conventional pulse oximeter monitors the perfusion of blood to the dermis and subcutaneous tissue of the skin. Because the skin is so richly perfused, it is relatively easy to detect the pulsatile component of the cardiac cycle. PPG signal contains information on the functioning of multiple organ systems and it has been used to measure blood pressure, respiration, depth of anesthesia and blood volume.

Traditional early warning scores (EWS) such as the National Early Warning Score (NEWS) have been used before to detect multiple life-threatening conditions in the hospital setting rcplondon.ac.uk/projects/outputs/national-early-warning-score-news-2. These scores are simple numerical models where input parameters are combinations of measured vital functions and clinical signs. Manual work is required in interpreting clinical signs and calculating the traditional EWS. Specialized interpretation of the score is also required when predicting different life-threatening conditions.

Convolutional neural network (CNN) is a supervised computerized model belonging to a class of deep neural networks. They were inspired by biological processes so that the connectivity pattern between neurons resembles the organization of the animal visual cortex. Individual cortical neurons respond to stimuli only in a restricted region of the visual field known as the receptive field. The receptive fields of different neurons partially overlap such that they cover the entire visual field. CNNs have applications in image and video recognition, recommender systems, image classification, medical image analysis, and natural language processing. CNN is a data driven method and it is able to learn common patterns from noisy input data related to training target. Given enough training data it should be able to learn optimal features from the noisy input data related to the specified training target. In contrast, traditional numerical methods used to analyze biosignals such as the ECG require extensive amounts of filtering and expert rules in order to create predictive models for each life-threatening condition.

Resnet is a convolutional neural network that can be utilized as a state of the art image classification model (web.archive.ora/web/20200815000000*/https://blog.roboflow.com/custom-resnet34-classification-model/ (Sep. 21, 2020)).

Tutorial on training a CNN to classify images: (web.archive.orq/web/20191023233024if /https://www.tensorflow.org/tutorials/images/c nn (Oct. 23, 2019)).

Creating and training a CNN model takes just a few lines of code (web.archive.org/web/20191208063212/https://www.tensorflow.org/tutorials/images/tran sfer learning (Oct. 31, 2019)).

Transfer learning is used to customize a model to a given task (web.archive.orq/web/20191208063212/https://www.tensorflow.org/tutorials/images/tran sfer learning (Dec. 8, 2019)).

Examples of CNN models for transfer learning comprise VGG, InceptionV3, and ResNet5 (web.archive.org/web/20181031030712if_/https://towardsdatascience.com/transfer-learning-from-pre-trained-models-f2393f124751 (31.10.2018)).

Transformer network, or a transformer, is a deep learning structure that makes few architectural assumptions about the relationship between its inputs, but that also scales to hundreds of thousands of inputs. The transformer network adopts the mechanism of attention, differentially weighing the significance of each part of the input data. Similarly to a CNN it can effectively learn the relationship of multiple very large input matrices related to the predicted target. An example of use of the transformer network, called Perceiver, for learning relationships between inputs is described in arxiv.org/pdf/2103.03206.pdf. These transformer networks can also be used in conjunction with transfer learning and implemented with few lines of code as described for the Perceiver that is a special case of a transformer network in pythonrepo.com/repo/lucidrains-perceiver-pytorch. Perceiver describes a transformer network that, similarly to a CNN, is able to recognize spatial relationships which are essential in understanding the concept of structured multi-modal inputs and their relationship to the predicted target. The Perceiver uses a cross-attention module to project an input high-dimensional byte array to a fixed-dimensional latent bottleneck before processing it using a stack of transformers in the low-dimensional latent space. The Perceiver iteratively attends to the input byte array by alternating cross-attention and latent transformer blocks.

US20180098739A1 discloses an early warning scoring system. The system comprises a computing device, a plurality of sensors for acquiring physiological signals from a patient, wherein the sensors are functionally connected to the computing device, and at least one alarm adapted to output an alert upon an early warning score (EWS) exceeding a predetermined level. The computing device receives the physiological signals from the sensors, analyzes the physiological signals, and based on the analyzed signals, calculates the early warning score, and compares the early warning score to predetermined limits and, if the score is outside the limits, triggers an alarm or actuates or modifies a treatment or medical intervention. An impedance measuring device measures impedance cardiography and impedance pneumography simultaneously. Preferably, the impedance data alone, or combined with one or more additional parameters are used to provide a diagnosis of a disease state. It was shown that for a given change in volume, laying supine yielded the greatest signal amplitude and lowest signal to noise during respiration. Digital signal processing measures such as filtering and oversampling as well as position of the subject can affect the noise in measurements.

### SUMMARY

The scope of protection sought for various embodiments of the invention is set out by the independent claims. The embodiments, examples and features, if any, described in this specification that do not fall under the scope of the independent claims are to be interpreted as examples useful for understanding various embodiments of the invention.

According to some aspects, there is provided the subject matter of the independent claims. Some further aspects are defined in the dependent claims. The embodiments that do not fall under the scope of the claims are to be interpreted as examples useful for understanding the disclosure.

### BRIEF DESCRIPTION OF THE DRAWINGS

The foregoing aspects and other features are explained in the following description, taken in connection with the accompanying drawings, wherein:

FIG. 1 is a block diagram of one possible and non-limiting system in which the example embodiments may be practiced.

FIG. 2 is a block diagram of a training device in accordance to at least some embodiments.

FIG. 3 illustrates block diagrams of detection devices in accordance to at least some embodiments.

FIG. 4 is a block diagram of one possible and non-limiting system in which the example embodiments may be practiced.

FIG. 5 illustrates an example of a method for a training device in accordance with at least some embodiments.

FIG. 6 illustrates an example of a method for a detection device in accordance with at least some embodiments.

FIG. 7 illustrates an example of a method for a training device in accordance with at least some embodiments.

FIG. 8 illustrates an example of a method for a training device in accordance with at least some embodiments.

FIG. 9 illustrates an example of a method for a training device in accordance with at least some embodiments.

FIG. 10 illustrates an example of a method for a training device in accordance with at least some embodiments.

FIG. 11 illustrates an example of a method for a training device in accordance with at least some embodiments.

FIG. 12 illustrates an example of a method for a detection device in accordance with at least some embodiments.

FIG. 13 illustrates an example of a method for a detection device in accordance with at least some embodiments.

FIG. 14 illustrates an example of a method for obtaining input data to a computerized model in accordance with at least some embodiments.

FIG. 15 illustrates an example of a method for obtaining input data for training a computerized model in accordance with at least some embodiments.

FIG. 16 illustrates an example of information indicating a life-threatening condition in accordance with at least some embodiments.

FIG. 17 illustrates an example of explanatory information for an output of a computerized model.

FIG. 18 illustrates an example of training a computerized model in accordance with at least some embodiments.

FIG. 19 illustrates time-domain sample sequences of biosignals in accordance with at least some embodiments.

FIG. 20 illustrates an example of information indicating at least two life-threatening conditions in accordance with at least some embodiments.

FIG. 21 illustrates an example of information indicating a differential diagnosis between multiple life-threatening conditions in accordance with at least some embodiments.

FIG. 22 illustrates an example of an alert display that may be displayed when a decision threshold for a life-threatening condition is reached for a particular patient in accordance with at least some embodiments.

FIG. 23 illustrates an example of a quality control display for at least two recorded and/or monitored biosignals in accordance with at least some embodiments.

### DETAILED DESCRIPTION OF EXAMPLE EMBODIMENTS

There is provided detection of one or more life-threatening conditions with the help of a computerized model. In order to detect a life-threatening condition by the computerized model, the computerized model is trained based on time-domain sample sequences of measurements of at least two biosignals from subjects and information indicating timing of the life-threatening condition. After the computerized model has been trained, measurements of at least two biosignals of a subject may be performed and the life-threatening condition may be detected by the trained computerized model based on time-domain sample sequences of the measurements. Training of the computerized model supports reliable detection of the life-threatening condition using - the trained computerized densities (2D PSDs) of the time-domain sample sequences are generated for detecting the life-threatening condition on the basis of spectral content of the biosignal measurements over time. In this context, the 2D PSD may be created by taking a FFT or STFT transformation from a time-domain sample sequence of a biosignal measurements where oversampling is presented in the length of the FFT (nperseg, nfft) as known sampling rate of the biosignal times oversampling factor. After the FFT transformation, the 2D PSD may be presented as two 2D matrices comprising the real and imaginary parts of the FFT transformation. Another way to present the 2D PSD is to take the absolute value of the FFT result and present the 2D PSD as one 2D matrix comprising the complex number distances. Additional abs and/or log-transformation may be applied to the different 2D PSD presentations to enhance details in the 2D matrix. Then, after the computerized model has been trained, 2D PSDs of measurement signals of at least two biosignals from a subject are generated and input to the trained computerized model. Processing of the measurement signals at the training of the computerized model and at detection, when the trained computerized model is used for detecting the life-threatening condition, provides 2D PSDs that have the same predefined characteristics, whereby training of the computerized model supports reliable detection of the life-threatening condition.

One or more described herein may be provided as standalone products, e.g. devices and/or software, or the examples may be provided as updates, e.g. software and/or hardware updates, to existing devices. Examples of the existing devices comprise at least commercially available patient monitoring devices, ventilators and anesthesia delivery systems.

In this context measurements of a vital function is a continuous biological measurement, i.e. a biosignal. Accordingly, two or more biosignals are obtained by measurements of at least two vital functions. The biosignal can be expressed as a time series of samples i.e. numerical or categorical value tied to a timestamp or a time-domain sample sequence. Measurements of a vital function or a biosignal may be non-invasive measurements or invasive measurements. It should be noted that the examples described herein may be applied to non-invasive measurements, invasive measurements or both non-invasive measurements and invasive measurements of biosignals.

In an example characteristics of the measurement signals obtained by the processing of the measurement signals at the training of the computerized model and at the detection comprise a number of samples of the 2D PSDs and a value range of the PSDs. Accordingly, the 2D PSDs input to the computerized model at training of the computerized model and at detection of the life-threatening condition may have the same number of samples and values from the same value range. In this way, fast training of the computerized model with a controlled data quality and reliable detection of the life-threatening condition using the trained computerized model may be supported.

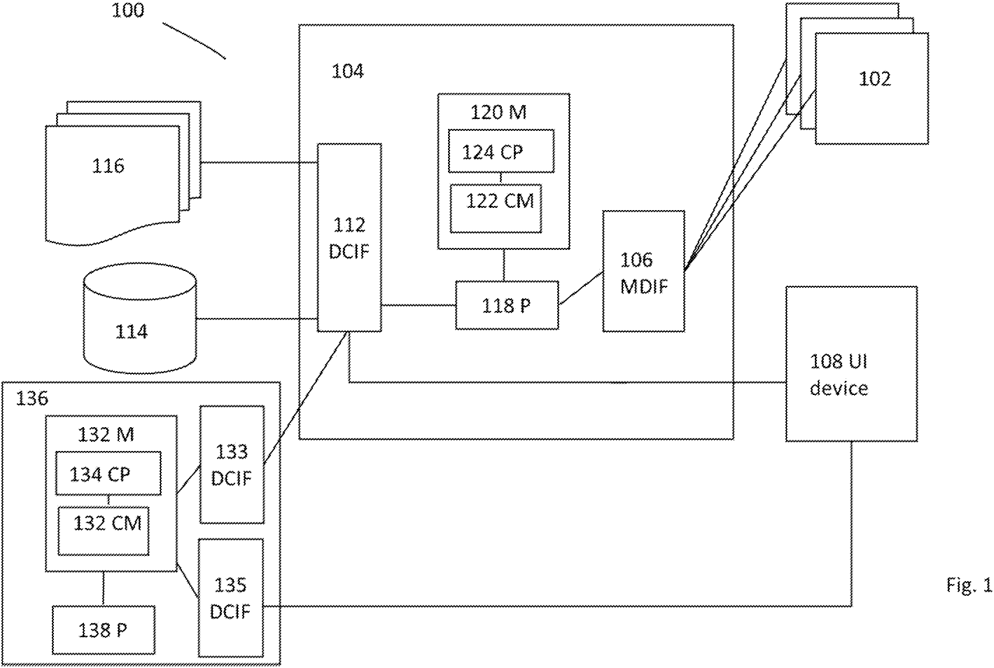

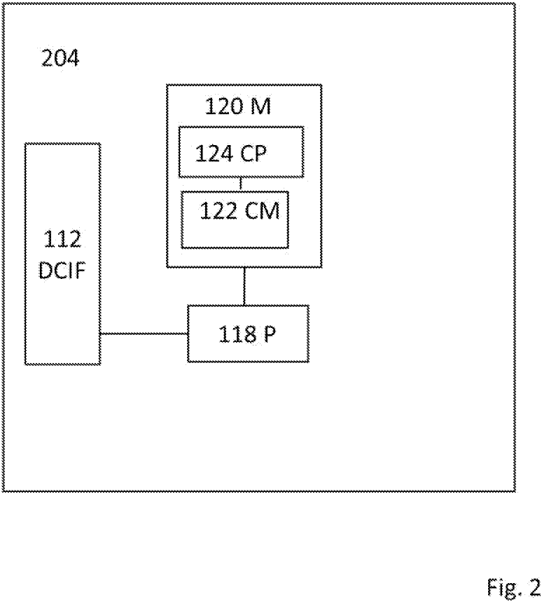

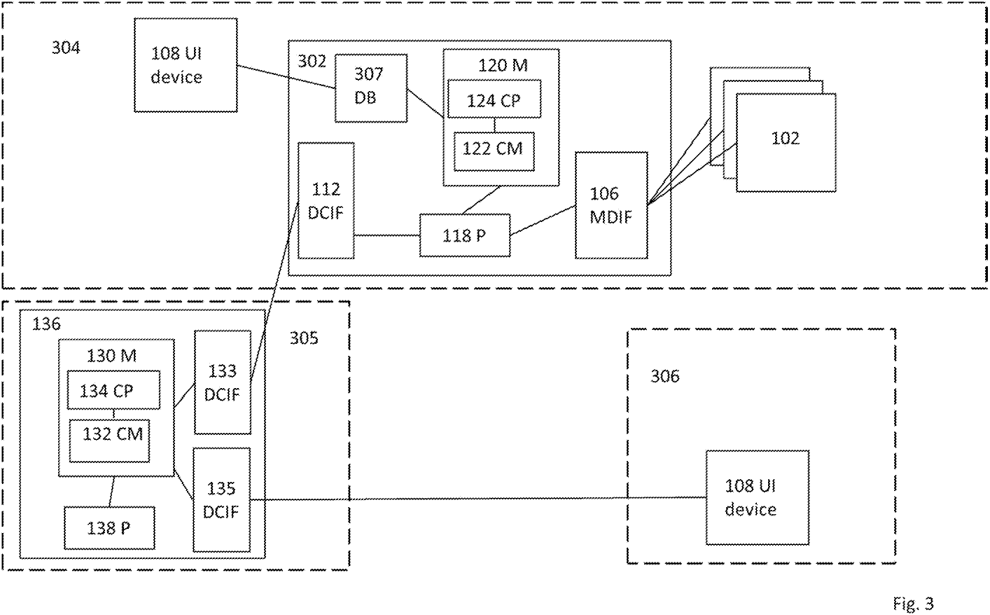

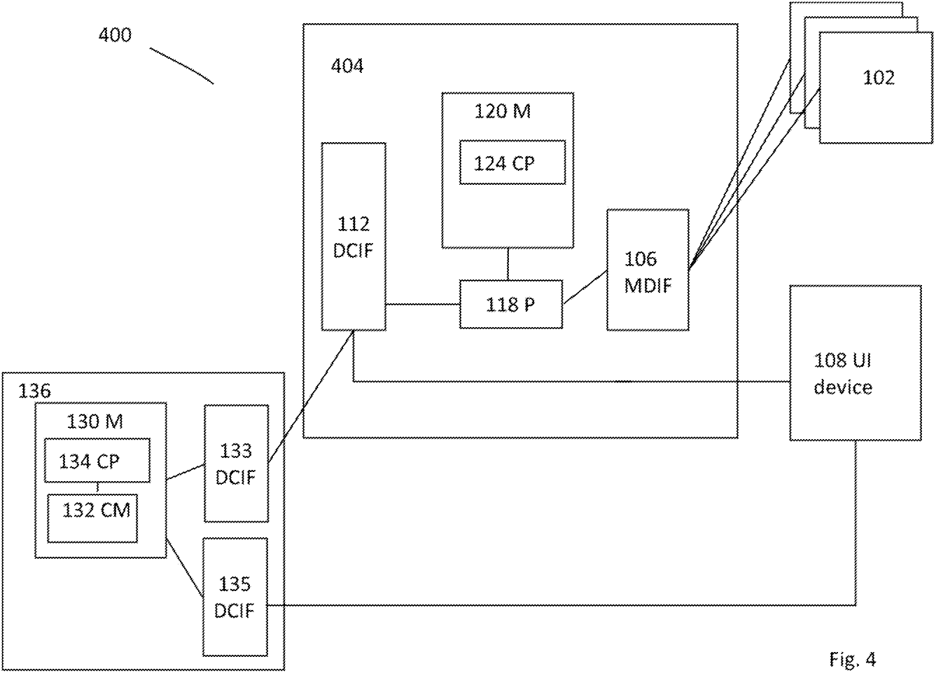

FIG. 1 is a block diagram of one possible and non-limiting system in which the example embodiments may be practiced. The system 100 of FIG. 1 may serve both for a detection device and a training device. FIG. 2 illustrates an example of a data processing device for training a computerized model (CM), i.e. a training device 204, with reference to components described with FIG. 1. FIG. 3 illustrates examples of data processing devices for detecting one or more life-threatening conditions using a computerized model, i.e. examples of detection devices 302, 304, 305, with reference to components described with FIG. 1. Accordingly, the training device 204 and detection device 304, 305 of FIG. 2 and FIG. 3 may be implemented by one or more components of a system in accordance with FIG. 1. On the other hand the training device 204 and detection device 302, 304, 305 of FIG. 2 and FIG. 3 may be separate devices, where a computerized model 122 may be trained by the training device 204 and after that the CM 122 may be installed to the detection device 302, 304, 305 for detecting life-threatening conditions. FIG. 4 illustrates an example of a system, where a data processing device 404 is provided without a computerized model in accordance with at least some embodiments. The system in FIG. 4 is described by components described with FIG. 1.

In accordance with at least some embodiments, a life-threatening condition comprises respiratory failure, sepsis, cardiac arrest, cardiac failure, congestive heart failure, renal failure, overhydration, pulmonary edema, hyper metabolic state, overexertion, brain injury, ischemic stroke, hemorrhagic stroke, multiorgan failure, anastomotic leak, internal bleeding, cardiac injury, intestinal obstruction, intestinal rupture, pulmonary embolus, opioid induced respiratory depression, seizure, over sedation, anaphylaxis, hypoxic brain damage, pneumonia, deep vein thrombosis, meningitis, malignant arrhythmia, hypovolemic shock, cardiogenic shock, obstructive shock, distributive shock, toxic shock, septic shock, myocardial infarction, wound infection, diabetic coma, endocarditis, myocarditis, pericarditis, intracranial hypertension, intestinal injury, liver failure, liver injury, pancreatitis, cardiovascular collapse, peritonitis, poisoning, drug reaction, aortic dissection and acute respiratory distress syndrome. The life-threatening condition may be detected based on measurements of biosignals.

Now referring to the system 100 of FIG. 1, the system comprises measurement devices 102 that may be connected to a data processing device 104 by one or more interfaces 106 that may be referred to measurement device interfaces (MDIFs). The MDIFs may be configured to connect the measurement devices. In this way the data processing device may obtain time-domain sample sequences of measurements from the measurement devices.

When device 104 is used in conjunction with one or more patient monitoring devices, an example of a MDIF is a patient monitoring device connector implementing the Datex-Ohmeda Record Specification (DRI) protocol. These data records can be accessed with a serial interface, called S/5 Computer Interface or with ethernet or wireless network connection using the S/5 Network Interface. Examples of patient monitoring device connector types are RS-232, MIB, WiFi, USB or RJ-45. When using the DRI protocol, the biosignal data can be requested with Application Data Interface (ADI) as waveform record. Each waveform record contains one or more subrecords, the types of which match the types specified in the waveform request, such as ECG or PPG biosignal. The waveform samples in different sub records inside the same waveform record represent the same time period but the waveform samples are of different biosignals, and therefore the waveform samples are of different waveform types. Because the different waveform types have different sample rates, only the first and last samples of different waveform types are in synchronization. With the S/5 Network Interface, it is not guaranteed that the different types of the waveform sub records are in the same record, and the requested waveforms may be split to separate records. For example, one record can contain waveform sub records ECG1 and ECG2 and another record sub record PPG. This can happen, for example, if more than 8 waveforms are requested from one monitor. In this case the synchronization between the waveform sub records can be implemented by using the r_time field defined in the DRI header. r_time is defined as the time when the record was transmitted. In this context, the time is defined as the number of seconds since 1.1.1970. Some compilers and libraries may use 1.1.1900 as the start moment of time. Waveform sub records are of variable length. The actual length of the sub record is included in the sub record itself, but the length can be calculated using the sub record offsets in the DRI header as well. When using S/5 Network Interface or S/5 Computer Interface, the timestamp field (r_time), known sampling rate of the waveform sub record and sub record length may facilitate the construction of a biosignal time series where each waveform value is linked to a real-world timestamp that can be related to the timing of a life-threatening event for a specific patient.

For example, when using the S/5 Computer Interface, all data from the monitor, and to the monitor, is transferred using flag-delimited frames. Each data frame starts and ends with a flag character. The value of the start and end flags is always 0×7E. As the application data may contain an arbitrary number of bytes with the same value, the following algorithm is used to detect the start and end of a frame correctly:
A control character (0×7D) is used to indicate the start of a control sequence.
At the transmitting end, each application data byte with value 0×7E (flag) or 0×7D (control character) is replaced with a control character and the original byte with the 5th (of bits 0-7) bit cleared.

This method guarantees that there are no flag characters in the outgoing application data stream. When a control character is received, it is not interpreted to be a part of the application data. The 5th bit of the next character must be set to restore the original value of the character. This conversion method is similar to that represented in the standard ISO/EI 13239:2002;. As the start and end flags are removed and necessary conversions done, the application data can be further processed. The application data consists of a DRI record and a checksum. When interfacing through the serial interface, each record is followed by a checksum byte. The checksum is calculated by summing all bytes in the DRI record using 8 bit unsigned arithmetic.

DRI can also be used to create a link between subject database 116 and a storage device 114 comprising time-domain sample sequences of measurements of at least two biosignals from subjects, or time-domain sample database, so that the subject database is constructed from Hospital Information System (HIS) using a common patient identifier that is also available from the patient monitor as Patient Information Message. Therefore providing an example how the recorded biosignal can be related to exactly one patient and to be temporally aligned to the timeline of one or more life-threatening events for the patient. The linking of a patient to a measured biosignal can also be performed statically when using the S/5 Computer Interface so that each patient monitor with serial port is assigned a unique identifier that is further stored, for example in the HIS, for each patient in addition to timestamps defining one or more segments in time when the patient has been monitored with the specific patient monitor.

It should be noted that the MDIF can also be implemented with internet protocols, such as REST, TCP or UDP or Internet of Things, IoT, protocols such as MQTT, Wi-Fi, ZigBee, Bluetooth, Bluetooth Low Energy, LoRa, Z-Wave, Low-power wide-area network, 5G IoT, NB-IoT or Sigfox.

The MDIF can also be interpreted as a direct wireless or wired connection to one or more measurement devices 102. For example, if device 104 is a patient monitoring device, single-board computer or a wearable device, the measurement device may transmit the measured biosignals as analog or digital values representing the temporal sequence of measurements performed by the measurement device. If required for the construction of time-domain sample database 114, the timestamps for the measurements and patient identifier linking the patient to subject database 116 can then be added at the processing device 104, thus creating the patient specific and temporally linked biosignal. It should be noted that the data processing device 104 may receive the patient identifier and trigger the start and stop of a recording for a specific patient identifier from an external system, such as HIS or UI device 108, via the DCIF 112, in order to create a patient specific biosignal. It should be noted that the data processing device 104 may synchronize its clock to external source using, for example a Network Time Protocol (NTP), via the DCIF in order to create a temporally linked biosignal.

The MDIF can also be interpreted as a combination of one or more patient monitoring devices, such as patient monitoring devices implementing the DRI protocol, and a direct wireless or wired connection to one or more measurement devices 102.

The data processing device 104 may provide at least one of training of a computerized model (CM) 122, recording subject database 116 and/or time-domain sample database 114 and detecting an increased risk for a life-threatening condition of a subject using the CM. The CM 122 may be trained to detect a plurality of life-threatening conditions. Each life-threatening condition may be trained on the basis of measurements of at least two biosignals from subjects. Examples of a data processing device 104 are a patient monitoring device, a general purpose computer, quantum computing device, cloud computing device, wearable device and a single-board computer.

The measurement devices 102 may be configured to measure biosignals from subjects. The measurements performed by the measurement devices may be received at the MDIFs, where the data processing device may access the measurements. It should be noted that in addition to communications of the measurements between the measurement devices and the data processing device 104, the interfaces may be configured to supply electric power to the measurement devices, whereby the measurement devices do not necessarily need their own power sources. It should be noted that when the data processing device 104 is connected to a patient monitoring device via MDIF, in addition to communications of the measurements between the measurement devices and the data processing device, the interfaces may be configured to supply electric power to the data processing device, whereby the data processing device do not necessarily need its own power source.

Suitable measurement devices 102 comprise at least measurement devices that are connected to patient monitoring devices. Examples of the measurement devices 102 in accordance with at least some embodiments comprise an electrocardiogram, ECG, signal measurement device, a thermocouple signal measurement device, electroencephalogram, EEG, signal measurement device, infrared signal measurement device, pressure signal measurement device, accelerometer signal measurement device, radar signal measurement device, ballistocardiographic signal measurement device, capnography signal measurement device, photoplethysmography signal measurement device, electrodermal activity signal measurement device, near-infrared spectroscopy signal measurement device, mid-infrared spectroscopy signal measurement device, transcutaneous bilirubin signal measurement device, impedance pneumography signal measurement device, electromyography, EMG, signal measurement device, magnetoencephalography, MEG, signal measurement device, electrogastrogram, EGG, signal measurement device, electrical impedance tomography, EIT, signal measurement device and an invasive blood pressure signal measurement device.

It should be noted that in accordance with at least some embodiments, a detection device 302 comprises an interface 106 configured to connect to a measurement device. Example of a detection device 302 is a patient monitoring device, a general purpose computer, a tablet, a smartphone, a single-board computer or a wearable computing device such as a smartwatch. Examples of the interfaces comprise at least one of an interface configured to connect to an electrocardiogram, ECG, signal measurement device, an interface configured to connect to a thermocouple signal measurement device, an interface configured to connect to electroencephalogram, EEG, signal measurement device, an interface configured to connect to a infrared signal measurement device, an interface configured to connect to a pressure signal measurement device, an interface configured to connect to a accelerometer signal measurement device, an interface configured to connect to a radar signal measurement device, an interface configured to connect to a ballistocardiographic signal measurement device, an interface configured to connect to a capnography signal measurement device, an interface configured to connect to a photoplethysmography signal measurement device, an interface configured to connect to a electrodermal activity signal measurement device, an interface configured to connect to a near-infrared spectroscopy signal measurement device, an interface configured to connect to a mid-infrared spectroscopy signal measurement device, an interface configured to connect to a transcutaneous bilirubin signal measurement device, an interface configured to connect to a impedance pneumography signal measurement device, an interface configured to connect to electromyography, EMG, signal measurement device, an interface configured to connect to magnetoencephalography, MEG, signal measurement device, an interface configured to connect to electrogastrogram, EGG, signal measurement device, electrical impedance tomography, EIT, signal measurement device and an interface configured to connect to an invasive blood pressure measurement device.

In accordance with at least some embodiments, examples of the time domain sample sequences of biosignals comprise an electrocardiogram, ECG, signal, a thermocouple signal, electroencephalogram, EEG, signal, infrared signal, pressure signal, accelerometer signal, radar signal, ballistocardiographic signal, capnography signal, photoplethysmography signal, electrodermal activity signal, near-infrared spectroscopy signal, mid-infrared spectroscopy signal, transcutaneous bilirubin signal, a impedance pneumography signal, EMG signal, magnetoencephalography, MEG, signal, electrogastrogram, EGG, signal, electrical impedance tomography, EIT, signal and an invasive blood pressure signal. Accordingly, the measurement devices may be configured to provide the data processing device 104 the time domain sample sequences or measurement signals that are processed by the processing device into time domain sample sequences. It should be noted that the ECG signal may be for measuring heart function. It should be noted that the EEG signal may be for measuring brain function. It should be noted that thermocouple signal may be for measuring body temperature. It should be noted that the pressure signal and accelerometer signal may be for measuring body movements and/or respiration. It should be noted that the radar signal may be for measuring body movements and/or respiration. It should be noted that the ballistocardiographic signal may be for measuring respiration and/or heart function. It should be noted that the capnography signal may be for measuring respiration, circulation and/or metabolism. It should be noted that the photoplethysmography signal may be for measuring oxygen saturation and/or blood pressure. It should be noted that the electrodermal activity signal may be for measuring nervous system activity. It should be noted that the near-infrared spectroscopy signal may be for measuring cerebral perfusion and/or blood glucose. It should be noted that the transcutaneous bilirubin signal may be for measuring non-invasive metabolic marker. It should be noted that the impedance pneumography signal may be for measuring respiration. It should be noted that electromyography signal may be for measuring muscle response or electrical activity in response to a nerve’s stimulation of the muscle. It should be noted that an invasive blood pressure signal may be for measuring intra-arterial blood pressure. It should be noted that magnetoencephalography, MEG, signal may be for measuring brain function. It should be noted that electrogastrogram, EGG, signal may be for measuring intestinal function. It should be noted that electrical impedance tomography, EIT, signal may be for measuring electrical conductivity, permittivity, and impedance of a part of the body inferred from surface electrode measurements.

It should be noted that the MDIFs 106 may comprise hardware and/or software for receiving time-domain sample sequences of measurements of the biosignals by the measurement devices 102. In an example, the data processing device 104 may comprise one or more MDIFs that may be implemented by software that defines a software interface for communications with the measurement devices for receiving time-domain sample sequences of measurements of the biosignals from the measurement devices. In an example, the data processing device 104 may comprise one or more MDIFs that may be implemented by physical connectors that connect the data processing device to the measurement devices by wired or wireless connections. In addition to the physical connectors, the data processing device may comprise software configured to process the measurements received from the physical connectors into time-domain sample sequences.

The data processing device 104 may comprise a data communications interface (DCIF) 112 for communications with one or more external systems and devices. Examples of external systems and devices comprise a storage device 114 comprising time-domain sample sequences of measurements of at least two biosignals from subjects, a subject database 116 storing computer-readable data of subjects, another data processing device 136, a computerized model 132 hosted on said another data processing device 136 and a user interface device 108. It should be noted that the user interface device may also be omitted from the system. An example implementation of a time-domain sample database 114 would be a folder in a file-system containing Hierarchical Data Format (HDF) data files. It should be noted that the data processing device 104 may also function as a recording device to facilitate the construction of time-domain sample database 114 and/or subject database 116 so that no training or detection is performed. During the recording phase the data processing device may take input from the 108 UI device or DCIF in order to start or stop recording for a specific patient identifier and/or record a life-threatening event and associated timestamp for a particular patient. These HDF data files are tagged, for example in the filename, separated by a static delimiter, with a unique patient identifier linking the specific HDF data file to exactly one patient stored in subject database 116. In addition to the unique patient identifier, the HDF files would be tagged with a unique biosignal identifier, so that the combination of patient identifier and biosignal identifier defines a continuous biosignal recording for exactly one patient. Example of a tagged HDF-file combining a patient identifier and a biosignal identifier would be 6783757_ECG.h5. Additionally, the HDF-file may be tagged with a start and stop timestamps that are presented in the file so a long continuous recording can be chunked to smaller segments for efficient storage and transfer. Example of a timestamp-tagged HDF-file would be 6783757_ECG_1633444663_1633744663.h5 where the two last delimited values present the UNIX-timestamps of start and stop times. The HDF-files may also be compressed in order to save bandwidth and/or disk space. The contents of an individual HDF file would then comprise a time series where a real-world timestamp, such as UNIX time, would be associated with a single biosignal sample value. The DCIF may be configured for data communications over a communications network or internally inside a computing device using for example a PCI Express bus providing access to a data storage device. The computer-readable data of subjects comprises information indicating timing of one or more life-threatening conditions of the subjects. An example implementation of a subject database 116 DCIF connection would be a Health Level Seven (HL7) connection to HIS, a connection to FTP-server, a connection to SSH-server, a connection to Network File System (NFS), a remote application programming interface, such as representational state transfer (REST), or a file in the local file-system such as a Comma-Separated Values (CSV)- or a spreadsheet file, e.g. a Microsoft Excel- file. The contents of the subject database would then indicate the timing of one or more life-threatening conditions of the subjects, or information where such timing can be inferred, for example the time of antibiotic administration due to suspicion of sepsis, one or more International Classification of Disease (ICD) codes specific for a life-threatening condition, SNOMED Clinical Terms, or resuscitation efforts due to cardiac arrest. It should be noted that the timing of one or more life-threatening conditions of the subjects and the timestamps in the time-domain sample database can be projected to the same temporal coordinate system so that the labeling of time-domain segments, or 2D PSDs of the biosignals, is possible in relation to a life-threatening event timestamp. The computer-readable data of subjects and the time-domain sample sequences of measurements of at least two biosignals from the subjects may be used for training the CM 122. The data communications provided by the DCIF may be based on the Internet protocols for example. An example implementation of a DCIF is a software interface or application programming interface configured to facilitate interaction between the data processing device and external systems and devices. An example of a DCIF application programming interface is a REST interface where data can be transferred using JSON-structures or as binary data such as HDF files. It should be noted that HDF files may also be transferred using Rsync-protocol via DCIF. HDF might be a preferred way to transfer biosignals as it provides a compact format for representing high-frequency time series data thus minimizing network load and data storage requirements. Another example of a DCIF application programming interface is a HL7 interface which may be preferred when communicating with a HIS. Examples of hardware implementation of the DCIF comprise a wireless communications module and a wired communications module. The DCIF may comprise one or more software components configured to control the hardware components. It should be noted that the data processing device 104 may transmit the result of a prediction of a life-threatening condition to an external system, such as HIS, using the DCIF.

The user interface device may provide a user interface via which input may be received from a user and/or information may be output to the user. Examples of the user interface devices 108 comprise displays or devices equipped with displays. The user interface device may be configured to connect to the DCIF for receiving information to be displayed and additionally communicating the data processing device user input received from a user of the display device. Examples of information received by the display device from the DCIF comprise at least information indicating one or more life-threatening conditions and information indicating an increased risk for a life-threatening condition. The display device may comprise or be connected to one or more user input devices that provide receiving user input. Examples of the user input devices comprise a touch screen, a keyboard, a computer mouse and a button.

It should be noted that the time-domain sample sequences of the subjects stored in the storage device 114 may be associated with records of the same subjects stored in the subject database. Preferably, the time domain sample sequences for each subject include samples for time periods without life-threatening conditions and samples for time periods with at least one life-threatening condition. Since the data in the storage device and the subject database is not restricted to data from only one subject or patient, but the data includes data from a plurality of subjects, the data supports training the CM to detect life-threatening conditions. In this way, the time domain sample sequences may facilitate training of the CM 122, 132 for detecting one or more life-threatening conditions.

The data processing device 136 may provide at least one of training of a computerized model (CM) 132 and detecting an increased risk for a life-threatening condition of a subject using the CM. The CM 132 may be trained to detect a plurality of life-threatening conditions. Each life-threatening condition may be trained on the basis of measurements of at least two biosignals from subjects.

Said another data processing device 136 may comprise one or more processors 138 and at least one memory (M) 130. The memory may store the computerized model (CM) 132 and computer program (CP) 134 operatively connected to the CM. The CP 134 may comprise instructions that when executed by the one or more processors 134 cause the data processing device 136 to perform one or more functionalities in accordance with a method described herein. In an example, the data processing device 136 may perform communications, or receiving, of information from the data processing device 104 for at least one of training of the computerized model 132 and detecting an increased risk for a life-threatening condition of a subject using the CM. The communicated information may comprise at least one of: time-domain sample sequences of measurements of at least two biosignals, windowed time-domain sample sequences and two-dimensional power spectral densities (2D PSDs) of the windowed time-domain sample sequences. A two-dimensional power spectral density of a time-domain sample sequence refers to a PSD that comprises distribution of power of the time domain-sample sequence into frequency components over time. Additionally, the data processing device 136 may perform communications of information from the data processing device 136 to the data processing device 104. The communicated information from the data processing device 136 to the data processing device 104 may comprise at least one of: information indicating a life-threatening condition based on 2D PSDs processed by the computerized model 132, information indicating an increased risk for a life-threatening condition on the basis of the output from the computerized model 132. In this way the data processing device 136 may take care of processing the time-domain sample sequences of measurements of at least two biosignals from subjects received by the data processing device 104 at the MDIFs from the measurement devices 102. It should be noted that the CM 132 may be trained similarly to CM 122 on the basis of computer-readable data of subjects and the time-domain sample sequences of measurements of at least two biosignals from the subjects. Since the CM 132 is connected to the DCIF of the data processing device 104, the training of the CM 132 may be controlled by the data processing device 104 using the computer-readable data of subjects and the time-domain sample sequences of measurements of at least two biosignals from the subjects.

In an example, the data processing device 136 may comprise one or more DCIFs 133, 135 for communications with at least one of the data processing device 104 and the user interface device 108. The DCIF 133 for communications with the data processing device may provide communications of comprise at least one of time-domain sample sequences of measurements of at least two biosignals, windowed time-domain sample sequences and two-dimensional power spectral densities (2D PSDs) of the windowed time-domain sample sequences. The DCIF 135 for communications with the user interface device 108 provides that information indicating a life-threatening condition and explanatory information may be communicated from the data processing device 136 to the user interface device to be displayed on a user interface. In an example, the DCIF 135 may comprise a Display Bus (DB) such as High-Definition Multimedia Interface (HDMI) or DisplayPort.

The data processing device 104 may comprise one or more processors (Ps) 118 and at least one memory (M) 120. The memory may store a computerized model (CM) and computer program (CP) 124 operatively connected to the CM 122. The CP may comprise instructions that when executed by the one or more processors cause the data processing device to perform one or more functionalities in accordance with a method described herein.

In an example the CP 124 may be connected to the CM 122 for input of data to the CM 122 and receiving data output by the CM. Examples of input data to the CM comprise two-dimensional Power Spectral Densities (2D PSDs) obtained from processing measurements of biosignals from subjects. Examples of output data from the CM comprise information indicating a life-threatening condition based on the 2D PSDs processed by the CM. It should be noted that additionally, the CP or another CP stored to the M may be configured to perform one or more control operations to the CM. Examples of the control operations comprise changing operating parameters of the CM and reading operating parameters of the CM.

In an example the CP 134 may be connected to the CM 132 for input of data to the CM 122 and receiving data output by the CM. Examples of input data to the CM comprise two-dimensional Power Spectral Densities (2D PSDs) obtained from processing measurements of biosignals from subjects. Examples of output data from the CM comprise information indicating a life-threatening condition based on the 2D PSDs processed by the CM. It should be noted that additionally, the CP or another CP stored to the M may be configured to perform one or more control operations to the CM. Examples of the control operations comprise changing operating parameters of the CM and reading operating parameters of the CM.

In accordance with at least some embodiments the CM 122, 132 comprises a convolutional neural network (CNN), a Bayesian convolutional neural network, an ensemble of convolutional neural networks, ensemble of Bayesian convolutional neural networks, a transformer network, an ensemble of transformer networks, Bayesian transformer network or an ensemble of Bayesian transformer networks. In an example changing operating parameters of the CM comprises changing weights of a convolutional neural network. In an example reading operating parameters of the CM comprises reading weights of a convolutional neural network. CNNs can have multiple 2D PSDs as inputs so information from multiple 2D PSDs can be processed simultaneously in the model and unique network weights can be learned from temporally aligned 2D PSD presentations in order to create risk models for a wide range of life-threatening conditions. CNN inputs can be fused early in the network and fed to a well-known template structure such as ResNet or each input can be fed to a separate well-known template to be merged in the output layer to facilitate the generation of a single probability value for the set of inputs. Template structure input can be also generated by sum or concatenation of the 2D PSDs. CNN can also be replaced with a transformer network as it is also able to effectively learn the relationship between multiple input 2D PSDs and the probability of a life-threatening condition. Having multiple 2D PSDs, .e.g. 2D PSDs generated based on different biosignals, as input, i.e. a multi-PSD input, to a CNN is advantageous for the CNN to learn concurrent spectral features present in signals measured from different organ systems when an organism is presenting with a life-threatening condition. This gives the CNN an example of a positive class of a defined life-threatening condition. Negative class of a defined life-threatening condition can be learned by the CNN based on a multi-PSD input comprising multiple PSDs measured from a control patient with uneventful measurement history related to the specific life-threatening condition. Multi-PSD input also helps in noise tolerance as features related to a specific life-threatening condition may be visible in multiple input PSDs at the same time, in both the positive class and the negative class. Therefore for example the effect of noisy PSD created from ECG measurement, i.e. ECG PSD, may have a minimal or at least very low effect on the prediction accuracy of sepsis as long as other input PSDs such as PSDs generated based on a PPG signal, i.e. PPG PSDs, have a clear signal. In this case the trained CNN may look for predictive features in the PPG PSD and ignore the noisy ECG PSD. Traditional CNN provides probability point estimates of a life-threatening condition based on the input PSDs. Clinical usability of the CM may be improved when using an ensemble of traditional CNNs to provide composite score of multiple CNNs or by using a Bayesian approach where probability of a life-threatening condition is not a point estimate but a distribution of plausible probability values. It should be noted that the transformer network may be used instead of the CNN(s) as explained above. Use of the transformer network may be preferred for providing flexibility and/or improved accuracy in learning relationships between input 2D PSDs. Transformer network may also be fed with sequences of input 2D PSDs in order to provide improved accuracy in predicting the target outcome by incorporating temporal change from consecutive 2D PSDs to the CM. The sequence of input 2D PSDs may have a length of 2, 3, 4, 5, 6 or higher number of 2D PSDs.

Referring now to FIG. 2 the training device 204 is described with components of the system 100 for training the CM 122 for detecting one or more life-threatening conditions. The training device may be a data processing device 104 described with FIG. 1 that is adapted for the purpose of training the CM. Therefore, detection of the life-threatening condition from live measurements of biosignals may not be performed by the training device 204. In an example use case, the training device 204 may be connected to the storage device 114 and the subject database 116 by the DCIF for receiving information indicating one or more life-threatening conditions and time-domain sample sequences of measurements of at least two biosignals associated with subjects. The received data from the storage device 114 and subject database may be used to train the CM 122. After training of the CM has been completed, the CM may be installed to the system 100 of FIG. 1, the dedicated detection device 304 described with FIG. 3 for detecting life-threatening conditions or to the system of FIG. 4.

Referring to FIG. 3, examples of detection devices 302, 304, 305 are described with components of the system 100 for detecting one or more life-threatening conditions using the CM 122, 132 trained by the system or the training device 204. In an example, a detection device 302, 304, 305 may be a data processing device 104 described with FIG. 1 that is adapted for the purpose of using the CM 122, 132 after it has been trained for detecting one or more life-threatening conditions. Therefore, training of the computerized model is not performed by the detection device 302, 304, 305. A device 306 is a display device that implements the functionality of UI device 108. The device may be connected to the detection device, e.g. at the DCIF 135. In this way information indicating a life-threatening condition and explanatory information may be communicated from the detection device to the device 306 and displayed on a user interface provided on the device 306. Detection device 304 may also comprise the functionality of the UI device 108 by the detection device comprising a UI device 108 connected to device 302 by a Display Bus (DB) 307 that is an example of a DCIF. An example of the DB 307 is a High-Definition Multimedia Interface (HDMI) or DisplayPort connector. The UI device 108 may be an external screen connected to the device 304 or the UI device, e.g. a screen, may be integrated to 304. It should be noted that the UI device may be omitted from the device 304. An example of detection device 304 is a patient monitoring device, a desktop computer, a laptop computer, a single-board computer, a tablet, a smartphone or a wearable device such as a smartwatch. An example of the detection device 305 is a separate computing device accessible from the network, using Internet protocols for example. It should be noted that the detection device 305 may be connected to one or more detection devices 304. An example of the display device 306 is, a tablet computer, a smartphone, a laptop computer, a wearable device such as a smartwatch, a single-board computer with a screen or a desktop computer with a screen. It should be noted that the display device 306 and/or UI device 108 may be omitted from the system. It should be noted that the display device 306 and/or UI device 108 may display information regarding the risk of life-threatening conditions for multiple patients and the display device may be connected to one or more detection devices 304, 305 in order to facilitate centralized monitoring, for example in a hospital ward or intensive-care unit. Accordingly, a detection device in accordance with at least some embodiments described herein may comprise at least one of the detection devices 302, 304, 305 or more than one of the detection devices 302, 304, 305 or the detection device may comprise all of the detection devices 302, 304, 305 It should be noted that at least when the data processing device 136 comprises a CM and is a part of a detection device or connected to the detection device, the CM may be omitted from the detection device.

Referring to FIG. 4, a system 400 comprises a data processing device 404 that is provided without a computerized model hosted locally at the data processing device. The system is described with components of the system 100 described with FIG. 1. The data processing device 404 may be operatively connected to the CM 132 that is hosted by the data processing device 136. It should be noted that the data processing device 136 may be connected to one or more processing devices 404. In an example use case, the data processing device 404 provides communications of information from the data processing device 404 to the data processing device 136, training of the computerized model 132 and/or detecting an increased risk for a life-threatening condition of a subject using the CM 132. The DCIF 133 provides that the CM hosted by the data processing device 136 may be used by the data processing device 404 for training of the CM and/or detection of the life-threatening conditions. Accordingly, the data processing device 404 may serve for at least one of a training device and a detection device. On the other hand, also the data processing device 136 may serve for at least one of a training device and a detection device. The communicated information from the data processing device 404 to the data processing device 136 may comprise at least one of time-domain sample sequences of measurements of at least two biosignals, windowed time-domain sample sequences and two-dimensional power spectral densities (2D PSDs) of the windowed time-domain sample sequences. The communicated information from the data processing device 136 to the data processing device 404 may comprise at least one of: information indicating a life-threatening condition based on 2D PSDs processed by the computerized model 132, information indicating an increased risk for a life-threatening condition on the basis of the output from the computerized model 132.

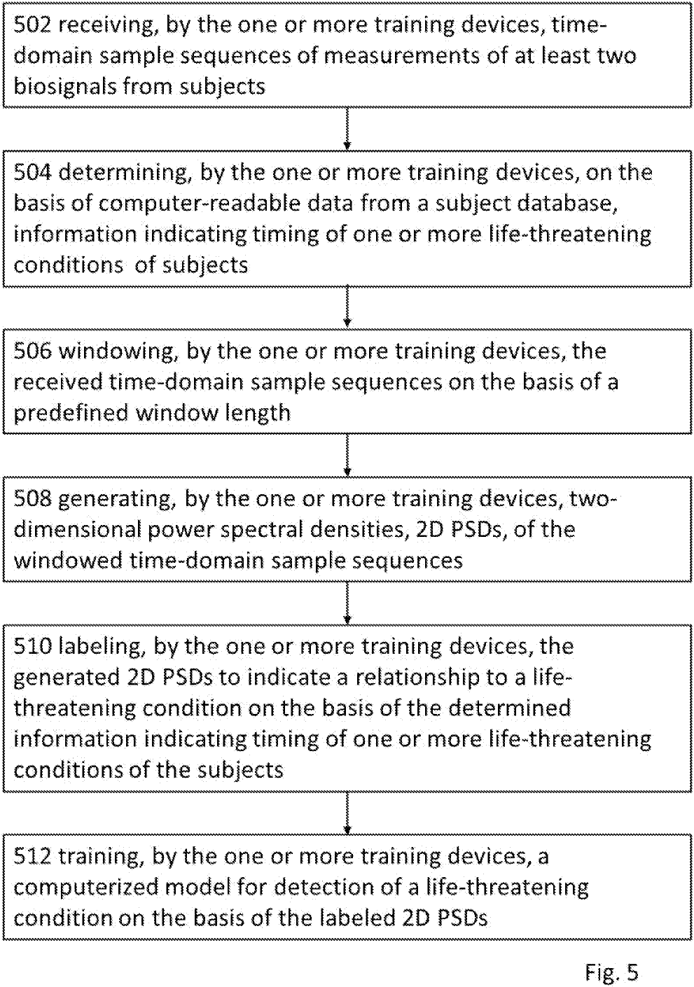

FIG. 5 illustrates an example of a method for a training device in accordance with at least some embodiments. The method may be performed by one or more data processing devices described with FIG. 1, FIG. 2 or FIG. 4 serving as a training device.

Phase 502 comprises receiving, by the one or more training devices, time-domain sample sequences of measurements of at least two biosignals from subjects. It should be noted that the biosignals may be measured concurrently from the subject and the biosignals may have different sampling rates.

Phase 504 comprises determining, by the one or more training devices, on the basis of the computer-readable data from a subject database, information indicating timing of one or more life-threatening conditions of the subjects.

Phase 506 comprises windowing, by the one or more training devices, the received time-domain sample sequences on the basis of a predefined window length.

Phase 508 comprises generating, by the one or more training devices, two-dimensional power spectral densities, 2D PSDs, of the windowed time-domain sample sequences.

Phase 510 comprises labeling, by the one or more training devices, the generated 2D PSDs to indicate a relationship to a life-threatening condition on the basis of the determined information indicating timing of one or more life-threatening conditions of the subjects.

Phase 512 comprises training, by the one or more training devices, a computerized model for detection of a life-threatening condition on the basis of the labeled 2D PSDs.

In an example phase 508 comprises applying log-transformations to the generated 2D PSDs and/or absolute value -transformations to the generated 2D PSDs. The computerized model may be trained in phase 512 based on the log-transformations of the generated 2D PSDs and/or the absolute value -transformations of the generated 2D PSDs. It should be noted that 2D PSD may be presented by two 2D matrices comprising the real and imaginary parts of an FFT transformation. The generated 2D PSDs may be fed to the CM in addition to the absolute value -transformations and/or the log-transformations of the 2D PSDs in phase 512 for training the CM.

In an example phase 508 comprises monitoring quality of one or more of the biosignals based on one or more biosignal-specific computerized models. The biosignal-specific computerized models may be trained specific to each biosignal. In this way automatic interpretation of a measured biosignal quality can be performed. For example, when a biosignal is obtained from a live measurement, the quality monitoring provides detecting a detached measurement probe. On the other hand if a biosignal is non-live data, data corruption may be detected based on the quality monitoring. Detection of insufficient and/or sufficient quality may be used to control a user interface, where a user interface element may be displayed corresponding to the detected quality.

In an example, phase 510 comprises that the computerized model is a transformer network, an ensemble of transformer networks, Bayesian transformer network or an ensemble of Bayesian transformer networks, and the method comprises: forming input sequences of the generated 2D PSDs; and training the computerized model by feeding the computerized model the formed sequences. In an example, sequences of 2D PSDs may be obtained based on following the phases 502 to 508 for generating 2D PSDs based on consecutive time windows, whereby the generated 2D PSDs form one or more sequences of 2D PSDs, that are consecutive in time. The sequences may comprise two or more, e.g. 3, 4, 5, 6, or higher number of PSDs. Using the sequences rather than individual 2D PSDs for the training provides that the CM may learn the relationship between the 2D PSDs in the sequence and the probability of a life-threatening condition. In an example, phase 504 comprises retrieving the computer readable data from a computer file or a database. The computer readable data may be e.g. medical records of the subjects and the computer readable data may include time instants of life-threatening conditions and/or elements whose association to one or more life-threatening conditions can be inferred, such as, diagnoses, treatments, procedures, SNOMED Clinical Terms and/or ICD-codes associated with timestamps. In an example, the computer readable data from a computer file or a database comprises one or more mappings of one or more of diagnoses, treatments, procedures, SNOMED Clinical Terms and/or ICD-codes to one or more life-threatening conditions of the subjects. In an example the mappings may be determined by clinical experts. For example, the instances of SNOMED diagnosis elements “Clinical sepsis” (ID: 447931005) and “Severe sepsis with acute organ dysfunction due to Meningococcus” (ID: 127311000119106) can be used to create mapping that connects these SNOMED classes to a life-threatening condition “Sepsis”. Time instants of diagnoses, treatments, procedures, SNOMED Clinical Terms and/or ICD-codes that map to life-threatening conditions of subjects may be each used separately for labeling in phase 504. On the other hand the time instants of diagnoses, treatments, procedures, SNOMED Clinical Terms and/or ICD-codes that map to life-threatening conditions of subjects may be combined into a time interval. Accordingly, the time interval may be determined based on a group of time instants of one or more diagnoses, treatments, procedures, SNOMED Clinical Terms and/or ICD-codes that map to a life-threatening condition of subject. In an example, each mapping of diagnoses, treatments, procedures, SNOMED Clinical Terms and/or ICD-codes to may be associated in addition to a time instant, also a time interval for one or more associated elements that comprises one or more of diagnoses, treatments, procedures, SNOMED Clinical Terms and/or ICD-codes. If the associated elements fall within the time interval associated with the mapping, the time interval may be extended to cover time instants of the associated elements.

In an example, phase 506 comprises selecting sets of samples from the time-domain sample sequences such that the sets represent data from different measurements of biosignals at substantially simultaneous time intervals. The samples may be selected by a windowing function.

In an example, phase 510 comprises that the labeling is performed according to a configuration that is specific to the life-threatening condition. For example, sepsis is a slowly evolving condition so 2D PSDs recorded hours after the sampling of a positive blood culture can still be labeled as positive for sepsis condition. However cardiac arrest may occur quickly and ECG measurement is interrupted by defibrillator shocks. Therefore labeling for cardiac arrest should not be performed on PSDs recorded after the detection of cardiac arrest. The labeling may be performed on the basis of user input via a user interface. The user may indicate via the user interface at least a time period and an endpoint of the time period. 2D PSDs representing biosignals during the time period may be then labeled to be associated with the life-threatening condition. Additionally the user may indicate via the user interface at least a time period and an endpoint of the time period where the patient is in a normal state to present a control condition. Labeling may also be performed automatically during the training phase by reading the timing of a life-threatening event from the patient database and labeling instructions for each life-threatening condition. These labeling instructions indicate at least one time period and an endpoint of the time period related to the timing of a life-threatening event where 2D PSDs are labeled as positive class or negative class for the life-threatening event in question. The time point, which decides if the specific 2D PSD is within the positive class or not, can be for example the start-time of the 2D PSD, the end-time of the 2D PSD or the midpoint of the 2D PSD. Labeling instructions can also contain patient identifiers that indicate a healthy or control patient recording. All 2D PSDs recorded from a control patient can then be used to present a negative class for the life-threatening event in question.

In an example, phase 510 comprises that the labeling indicates at least one of a presence of the life-threatening condition within the 2D PSD and relevancy of the 2D PSD for determining the life-threatening condition.

In an example, phase 512 comprises that the CM is fed the generated 2D PSDs. The generated 2D PSDs may be presented by 2D matrices comprising the real and imaginary parts of the FFT transformation. Another way to present the 2D PSD is to take the absolute value of the FFT result and present the 2D PSD as one 2D matrix comprising the complex number distances. Additional absolute value -transformation and/or log-transformation may be applied to the different 2D PSD presentations to enhance details in the 2D matrix.

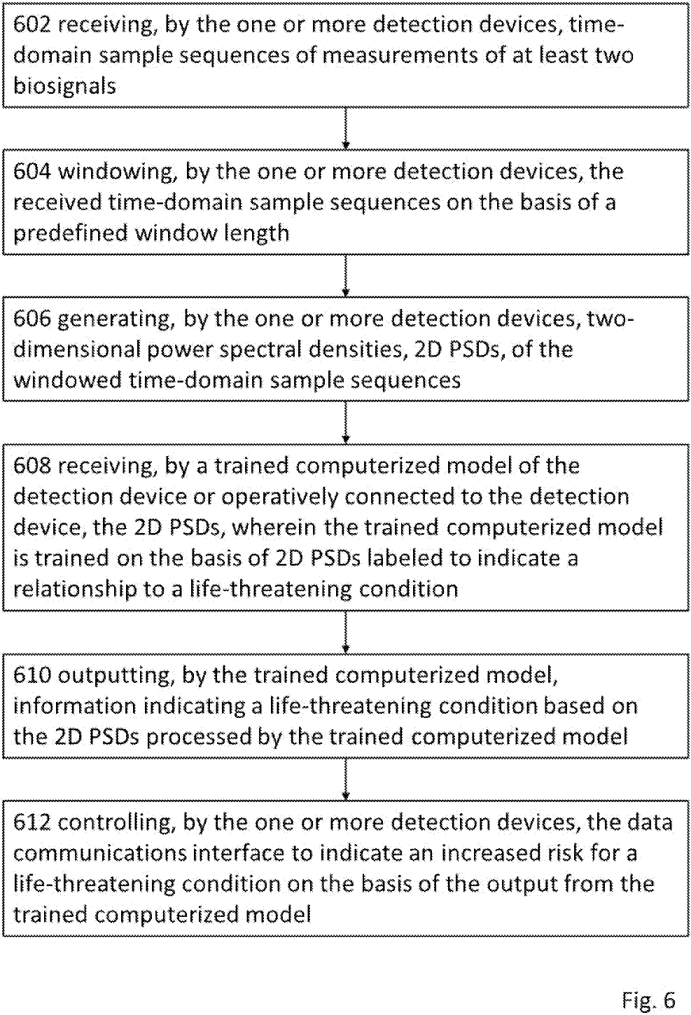

FIG. 6 illustrates an example of a method for a detection device in accordance with at least some embodiments. The method may be performed by one or more data processing devices described with FIG. 1 or FIG. 3 or FIG. 4 serving as a detection device.

Phase 602 comprises receiving, by the one or more detection devices, time-domain sample sequences of measurements of at least two biosignals from a subject. It should be noted that the biosignals may be measured concurrently from the subject and the biosignals may have different sampling rates.

Phase 604 comprises windowing, by the one or more detection devices, the received time-domain sample sequences on the basis of a predefined window length.

Phase 606 comprises generating, by the one or more detection devices, two-dimensional power spectral densities, 2D PSDs, of the windowed time-domain sample sequences. Here the 2D PSD generation may include additional preprocessing steps such as oversampling as described in 802.

Phase 608 comprises receiving, by a trained computerized model of the detection device or operatively connected to the detection device, the 2D PSDs, wherein the trained computerized model is trained on the basis of 2D PSDs labeled to indicate a relationship to a life-threatening condition. Here the trained computerized model may include additional preprocessing operations learned during the training such as scaling, oversampling or combining multiple 2D PSDs as described in 702, 802 and 902.

Phase 610 comprises outputting, by the trained computerized model, information indicating a life-threatening condition based on the 2D PSDs processed by the trained computerized model.

Phase 612 comprises controlling, by the one or more detection devices, the data communications interface to indicate an increased risk for a life-threatening condition on the basis of the output from the trained computerized model.

In an example phase 606 comprises applying log-transformations to the generated 2D PSDs and/or absolute value transformations to the generated 2D PSDs. The log-transformations of the generated 2D PSDs and/or the absolute value -transformations of the generated 2D PSDs may be fed to the computerized model in phase 608. It should be noted that 2D PSD may be presented by two 2D matrices comprising the real and imaginary parts of an FFT transformation. The generated 2D PSDs may be fed to the CM in addition to the absolute value -transformations and/or the log-transformations of the 2D PSDs for detecting the increased risk for a life-threatening condition on the basis of the output from the trained computerized model.

In an example, phase 606 comprises that the generated 2D PSDs may be presented by 2D matrices comprising the real and imaginary parts of the FFT transformation. Another way to present the 2D PSD is to take the absolute value of the FFT result and present the 2D PSD as one 2D matrix comprising the complex number distances. Additional absolute value -transformation and/or log-transformation may be applied to the different 2D PSD presentations to enhance details in the 2D matrix.

In an example phase 606 comprises monitoring quality of one or more of the biosignals based on one or more biosignal-specific computerized models. The biosignal-specific computerized models may be trained specific to each biosignal. In this way automatic interpretation of a measured biosignal quality can be performed. For example, when a biosignal is obtained from a live measurement, the quality monitoring provides detecting a detached measurement probe. Detection of insufficient and/or sufficient quality may be used to control a user interface, where a user interface element may be displayed corresponding to the detected quality. Additionally, the detected quality may be used to determine a reliability of a detected life-threatening condition or a reliability of an indication thereof. Accordingly, the user interface element may be displayed in connection with information indicating the detected life-threatening condition which helps the user understand whether the results of the detection of the life-threatening conditions has been negatively impacted by biosignal quality.

In an example in accordance with at least some embodiments, phase 608 comprises that the computerized model is a transformer network, an ensemble of transformer networks, Bayesian transformer network or an ensemble of Bayesian transformer networks, and the method comprises: forming input sequences of the generated 2D PSDs; and feeding the formed sequences to the computerized model. In an example, sequences of 2D PSDs may be obtained based on following the phases 602 to 606 for generating 2D PSDs based on consecutive time windows, whereby the generated 2D PSDs form one or more sequences of 2D PSDs, that are consecutive in time. The sequences may comprise two or more, e.g. 3, 4, 5, 6, or higher number of PSDs. Using the sequences rather than individual 2D PSDs as input to the CM provides that the CM may learn relationships between the 2D PSDs of each input sequence for detecting the increased risk for a life-threatening condition in phase 612.

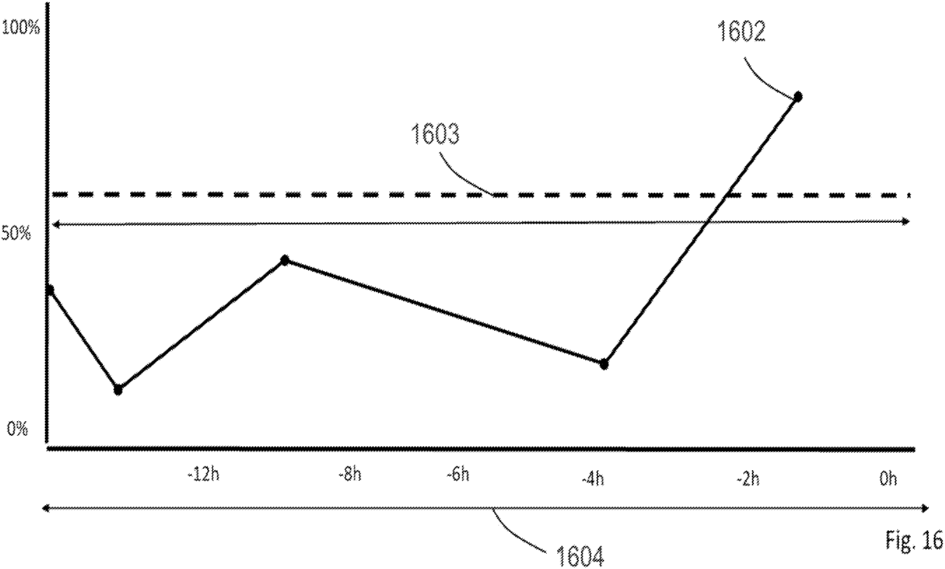

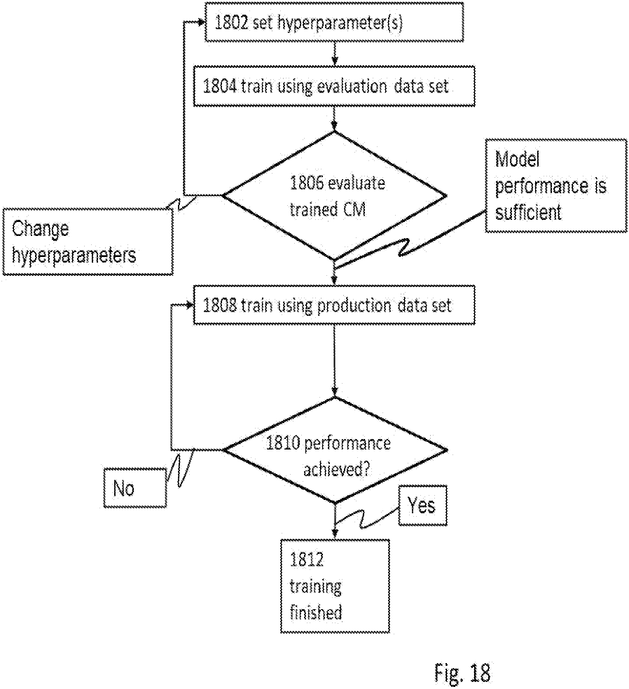

In an example in accordance with at least some embodiments, the phases 602 to 612 may be performed by one or more training devices instead of the detection devices or one or more training devices capable of serving both for training devices and detection devices. The phases 602 to 612 may be performed after completion of the training of the computerized model in accordance with the method of FIG. 5, or for evaluating whether the training of the computerized model has been completed. Indication of the increased risk provided by phase 612 may be evaluated against reference data for evaluating whether the training has been completed. Indication of the increased risk provided by phase 612 may also be used to calculate CM performance metrics, optimize hyperparameters in FIG. 18 and calculate a decision threshold 1603 in FIG. 16 using a reference patient database.

In an example phase 604 comprises that the window length is a predefined number of samples that may be different for each time-domain sample sequence as the concurrently measured biosignals may have different sampling rates.

In an example in phase 612, the information indicating a life-threatening condition may comprise information for a time interval from the present time to past or future time. In this way the potential of the life-threatening condition may be evaluated before the life-threatening condition actually takes place or the temporal history of the risk of a life-threatening condition observed.

In an example phase 612 comprises that the information indicating a life-threatening condition comprises one or more values indicating a risk for the life-threatening condition. The values may be given for a time interval from the present time to past or future time. In this way the potential of the life-threatening condition may be evaluated. It should be noted that the values may be median filtered values or Kalman filtered values over the time interval to reduce effect of single high peak values and in this way facilitating evaluation of the risk for the life-threatening condition.

In an example phase 612 comprises that an increased risk is determined on the basis of one or more values output by the computerized model indicating a risk for the life-threatening condition. The values may be given for a time interval from the present time to past or future time. It should be noted that the values may be median filtered values or Kalman filtered values over the time interval to reduce effect of single high peak values and in this way facilitating evaluation of the risk for the life-threatening condition. Raw values for the risk or filtered values for the risk may be compared with a threshold for establishing an increased risk for the life-threatening condition.

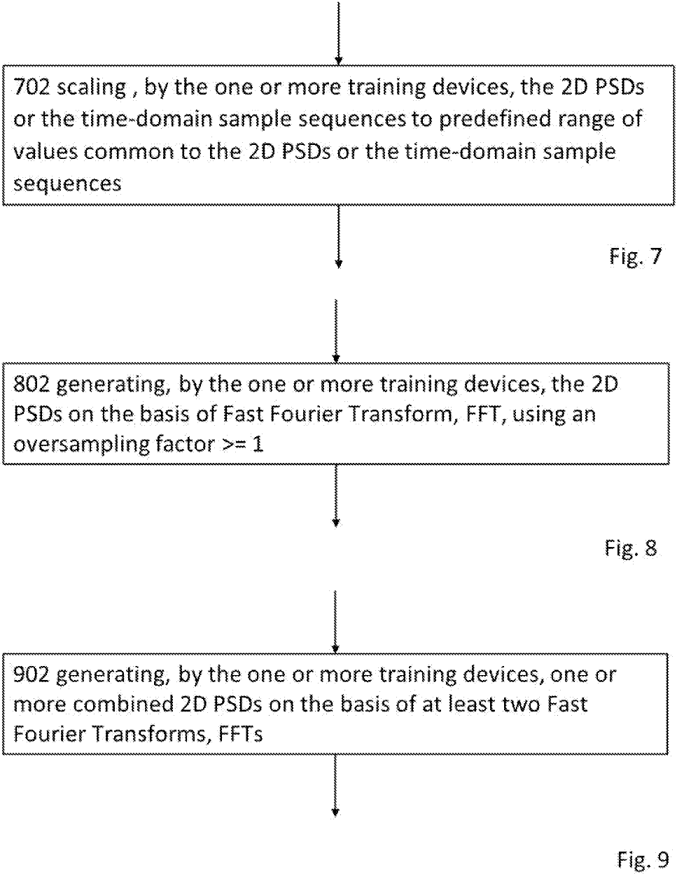

FIG. 7 illustrates an example of a method for a training device in accordance with at least some embodiments. The method may be performed by one or more data processing devices described with FIG. 1, FIG. 2 or FIG. 4 serving as a training device in connection with one or more of the phases of FIG. 5.

Phase 702 comprises scaling, by the one or more training devices, the 2D PSDs or the time-domain sample sequences to predefined range of values common to the 2D PSDs or the time-domain sample sequences. The scaling provides that values of samples of the time-domain sample sequences or the 2D PSDs may be adapted to the predefined range, whereby compatibility of time domain sample sequences from different manufacturers of measurement devices may be supported. On the other hand, scaling the 2D PSDs, provides that the scaled 2D PSDs may be compared with each other. Scaling of the 2D PSDs may be preferred over scaling of the time-domain sample sequences since conversion between the time-domain and frequency domain may be performed using non-scaled values, thereby ensuring accuracy of the frequency-domain information. Scaling also helps in normalizing the PSDs to a standard presentation when device specific calibration or amplification settings are present.

In an example, phase 702 comprises interpolating or decimating the 2D PSDs to a constant height and width. The height and width of the 2D PSD refer to dimensions of the 2D PSD in frequency and time domain, e.g. numbers of samples in frequency and time domains, of the 2D PSDs in time and frequency domain. The scaling may be performed regardless of oversampling factor or time sample sequence length of the 2D PSDs.

FIG. 8 illustrates an example of a method for a training device in accordance with at least some embodiments. The method may be performed by one or more data processing devices described with FIG. 1, FIG. 2 or FIG. 4 serving as a training device in connection with one or more of the phases of FIG. 5.

Phase 802 comprises generating, by the one or more training devices, the 2D PSDs on the basis of Fast Fourier Transform, FFT, using an oversampling factor ≥ 1. The oversampling factor ≥1 provides that the length of the FFT is greater than the sampling frequency for a given time-domain sample sequence. Oversampling of the sample sequences facilitates the detection of faint frequency components unique to specific life-threatening condition. Without oversampling these components could be hidden in noise at the resulting 2D PSDs.

FIG. 9 illustrates an example of a method for a training device in accordance with at least some embodiments. The method may be performed by one or more data processing devices described with FIG. 1, FIG. 2 or FIG. 4 serving as a training device in connection with one or more of the phases of FIG. 5.

Phase 902 comprises generating, by the one or more training devices, one or more combined 2D PSDs on the basis of at least two Fast Fourier Transforms, FFTs. The FFTs may generate 2D PSDs. The 2D PSDs may be generated based on subsequent time-domain sample sequences. The 2D PSDs may be combined in time, whereby the combined 2D PSD shows a frequency domain representation over the combined time intervals of the 2D PSDs. On the other hand the 2D PSDs may be generated based on the same time-domain sample sequence using different or the same FFTs. In this case, the generated 2D PSDs may be combined in frequency to provide a combined 2D PSD that comprises spectral information from a wider range than the individual FFTs. Advantage of this is to be able to use differently sized input data when reading the sample sequences and generating a standard 2D PSD presentation that the CM has been trained to interpret.

In accordance with at least some embodiments, phase 902 comprises that the FFTs have the same oversampling factors or the oversampling factors of the FFTs of the single 2D PSDs are different. Accordingly, the training devices may be configured to perform two or more FFTs that have different oversampling factors. The FFTs may be adapted for different frequency ranges by using a different oversampling factor for different frequency ranges. Accordingly, each time domain sequence may be processed by at least two FFTs that have different oversampling factors ≥1. In this way two 2D PSDs may be generated that may be combined in frequency to obtain a combined 2D PSD, where the combined 2D PSD has been adapted for at least two frequency ranges. Advantage of this is that for a specific sample sequence it may be more important to have a high oversampling factor to detect faint but slowly changing features in the PSD. At the same time the other PSD could have a smaller oversampling factor in order to detect faster changes but with reduced signal to noise ratio. Combination of these two differently sampled PSDs would then provide the CM the ability to detect both kind of features.

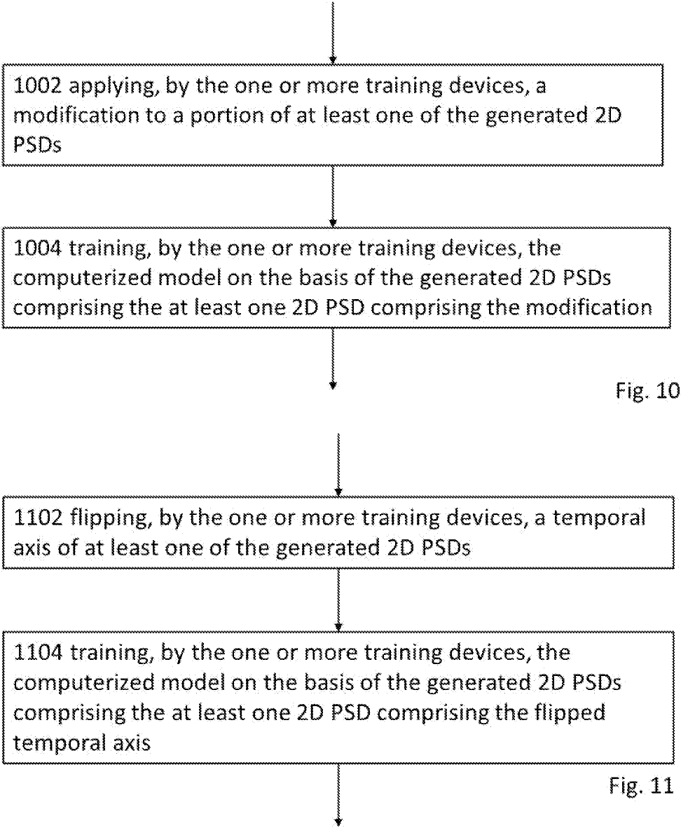

FIG. 10 illustrates an example of a method for a training device in accordance with at least some embodiments. The method may be performed by one or more data processing devices described with FIG. 1, FIG. 2 or FIG. 4 serving as a training device in connection with one or more of the phases of FIG. 5.

Phase 1002 comprises applying, by the one or more training devices, a modification to a portion of at least one of the generated 2D PSDs.

Phase 1004 comprises training, by the one or more training devices, the computerized model on the basis of the generated 2D PSDs comprising the at least one 2D PSD comprising the modification. The modification of the 2D PSDs provides that the computerized model is trained for corrupted, noisy, deficient or otherwise non-ideal values of input data and detection of life-threatening condition may be facilitated even with distorted data and in non-ideal conditions.

In an example phase 1002, comprises that the modification comprises at least one of adding noise and blanking the portion of at least one of the 2D PSDs.

In an example, phase 1002 comprises that 2D PSDs may be input to the computerized model without modification and also with one or more modifications. Accordingly, the computerized model may be input both a modified 2D PSD and an unmodified version of the 2D PSD. The labeling of the 2D PSDs supports the CM learning to detect a life-threatening condition from noisy and incomplete 2D PSDs making it more robust in identifying the life-threatening condition from real signals that are rarely ideal.

FIG. 11 illustrates an example of a method for a training device in accordance with at least some embodiments. The method may be performed by one or more data processing devices described with FIG. 1, FIG. 2 or FIG. 4 serving as a training device in connection with one or more of the phases of FIG. 5.

In accordance with at least some embodiments, phase 1102 comprises flipping, by the one or more training devices, a temporal axis of at least one of the generated 2D PSDs.

In accordance with at least some embodiments, phase 1104 comprises training, by the one or more training devices, the computerized model on the basis of the generated 2D PSDs comprising the at least one 2D PSD comprising the flipped temporal axis. The flipping of the 2D PSD provides that the values of the input data are not changed, while the computerized model may be trained with corrupted input data. The labeling of the 2D PSDs supports the computerized model learning to detect a life-threatening condition based on the flipped and non-flipped 2D PSDs input to the computerized model as the flipping provides an example of important feature in the 2D PSD happening at different time than in the real recording. This adds more variance to the training data and makes the CM more robust in extracting meaningful information regardless of timestamp of the recorded feature. In an example, in phase 1104, 2D PSDs may be input to the computerized model without flipping and also with flipping. The labeling of the 2D PSDs supports the computerized model learning to detect a life-threatening condition based on the flipped and non-flipped 2D PSDs input to the computerized model.

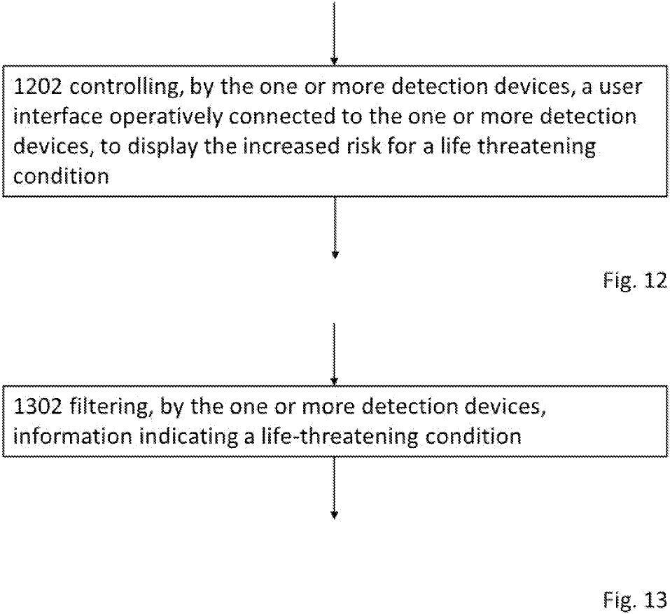

FIG. 12 illustrates an example of a method for a detection device in accordance with at least some embodiments. The method may be performed by one or more data processing devices described with FIG. 1, FIG. 3 or FIG. 4 serving as a detection device in connection with one or more of the phases of FIG. 5.

In accordance with at least some embodiments, phase 1202 comprises controlling, by the one or more detection devices, a user interface operatively connected to the one or more detection devices, to display the increased risk for a life-threatening condition. An operator of the detection device may learn from the displayed increased risk that there is a likelihood that the subject would need a treatment, a treatment plan of the subject would need to be updated and/or one or more operational parameters treatment of treatment machines connected to the subject would need to be adjusted to avoid the life-threatening condition.

FIG. 13 illustrates an example of a method for a detection device in accordance with at least some embodiments. The method may be performed by one or more data processing devices described with FIG. 1, FIG. 3 or FIG. 4 serving as a detection device in connection with one or more of the phases of FIG. 5.

In accordance with at least some embodiments, phase 1302 comprises filtering information indicating a life-threatening condition. The information indicating a life-threatening condition may be output by a computerized model. The information indicating a life-threatening condition may comprise a risk of a life-threatening condition for a subject. The risk may comprise values given for a time interval. The time interval may be e.g. from a present time, past time or a future time. The values may be filtered, e.g. by median filtering or Kalman filtering to reduce the effect of single high peak values and/or dips. In this way, when the risk is evaluated by a user, e.g. when the risk is displayed on a display device, the high peak values and low dips may be evened out thereby making it easier for the user to arrive in an overall estimation of the situation regarding the life-threatening condition of the subject.

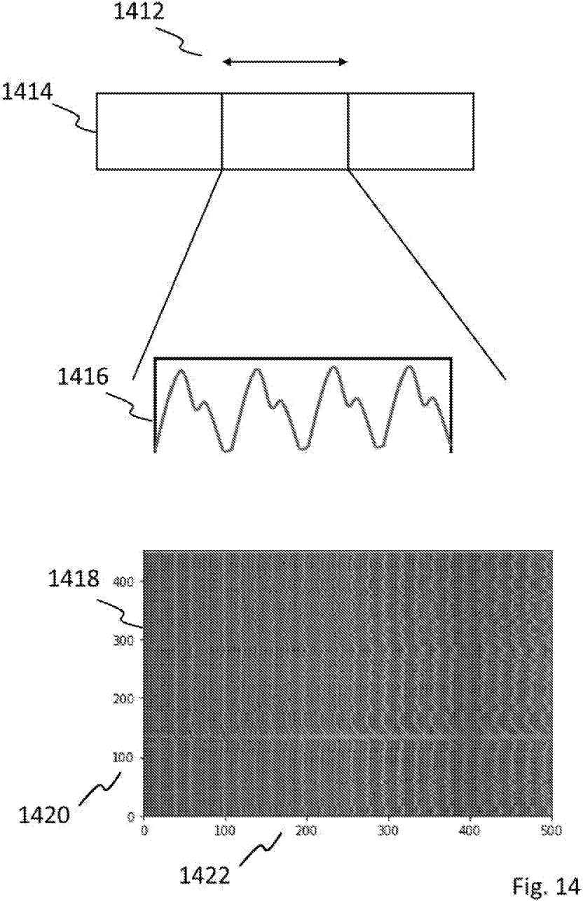

FIG. 14 illustrates an example of a method for obtaining input data to a computerized model in accordance with at least some embodiments. The method may be performed by one or more data processing devices described with FIG. 1, FIG. 2, FIG. 3 or FIG. 4 serving as a detection device or a training device in connection with one or more of the phases of FIG. 5 or FIG. 6. The input data may comprise PSDs generated based on time-domain sample sequences of measurements of at least two biosignals from one or more subjects.

A time-domain sample sequence 1414 may be obtained by a measurement of a biosignal from a subject e.g. as described at 506 and 602. The time-domain sample sequence may be windowed into sets 1416 of samples that represent a predefined time interval 1412. It should be noted that the time window can also be translated to the number of samples for each biosignal as the sample rates of the signals are known. The windowing provides that time intervals represented by sets of samples from time-domain sample sequences from different measurements of biosignals are windowed at substantially simultaneous time intervals. The samples may be selected by a windowing function. A 2D PSD 1418 may be generated on the basis of the windowed time-domain sample sequence. The PSD may be represented in a two-dimensional graph comprising a frequency axis 1422 and a time axis 1420. Accordingly, a 2D PSD may be generated on the basis of each window of samples or set of samples. 2D PSDs may represent different biosignal measurements. Accordingly, 2D PSDs may be generated based on windowed sample sequence from each biosignal measurement.

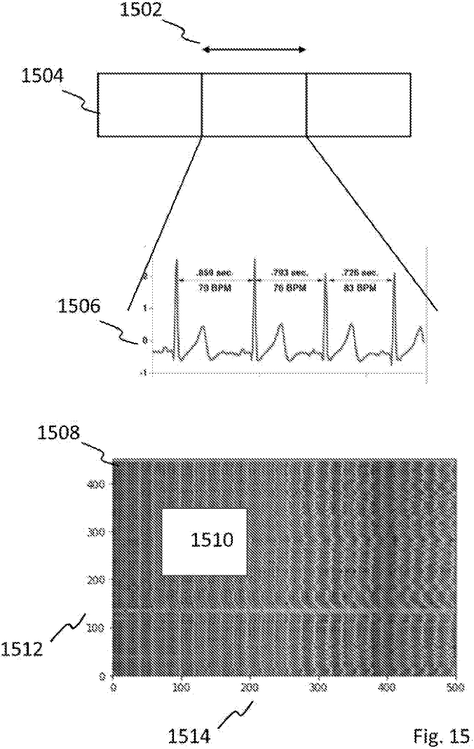

FIG. 15 illustrates an example of a method for obtaining input data for training a computerized model in accordance with at least some embodiments. The method may be performed by one or more data processing devices described with FIG. 1, FIG. 2 or FIG. 4 serving as a training device in connection with one or more of the phases of FIG. 5. The input data may comprise PSDs generated based on time-domain sample sequences of measurements of at least two biosignals from one or more subjects.

A time-domain sample sequence 1504 may be obtained by a measurement of a biosignal from a subject e.g. as described at 506. The time-domain sample sequence may be windowed into sets 1506 of samples that represent a predefined time interval 1502. The windowing provides that time intervals represented by sets of samples from time-domain sample sequences from different measurements of biosignals are windowed at substantially simultaneous time intervals. The samples may be selected by a windowing function. A 2D PSD 1508 may be generated on the basis of the windowed time-domain sample sequence. The PSD may be represented in a two-dimensional graph comprising a frequency axis 1514 and a time axis 1512. Accordingly, a 2D PSD may be generated on the basis of each window of samples or set of samples. 2D PSDs may represent different biosignal measurements. Accordingly, 2D PSDs may be generated based on windowed sample sequence from each biosignal measurement. A modification 1510, may be applied to a generated 2D PSD in accordance with 1002 and/or a temporal axis 1512 of the generated 2D PSD may be flipped in accordance with 1102.

FIG. 16 illustrates an example of information indicating a life-threatening condition in accordance with at least some embodiments. The information may be a graph displayed on a user interface of a display device. It should be noted that multiple graphs can be presented for a single patient in order to concurrently indicate the risk for multiple life-threatening conditions. The information indicating a life-threatening condition may comprise a risk of a life-threatening condition for a subject. The risk may be represented by values 1602 given for a time interval 1604 from the present time to past in order to observe current and historical values of the risk of a life-threatening condition. Accordingly, the risk value of the life-threatening condition is presented as the right most value in the representation for the present time. The values may be filtered, e.g. by median filtering or Kalman filtering to reduce effect of single high peak values and/or dips. 1603 represents a predefined decision threshold where the risk of a life-threatening condition reaches a specific sensitivity and specificity value. This threshold helps the care staff to decide when to take action in order to prevent the life-threatening condition. When the values 1602 exceed the predefined threshold 1603, an increased risk for a life-threatening condition may be determined by one or more detection devices or a user interface connected to the detection devices, in accordance with at least some embodiments. The predefined threshold 1603 may be set on the basis of testing data from a testing database compromising both case and control patients for the life-threatening condition in question. The decision threshold 1603 may be set for example, to a clinically meaningful combination of sensitivity and specificity values for a particular life-threatening condition, or it can be heuristically set to an optimal value, for example maximizing the value of a Youden’s J statistic on a validation data set. The testing data may be fed to the trained computerized model and the predefined threshold may be set to a desired value which corresponds to sensitivity and specificity of detecting the life-threatening condition in the evaluation data. It is also possible to calibrate the CM output with a representative testing dataset comprising biosignals of both case and control patients and biosignals measured from these patients. The calibration of the model output is made by adjusting the positive weight coefficient in the loss function during training so that a desired trade off between recall (also known as sensitivity) and precision (also called positive predictive value) is achieved by adding weights to positive class labels. An example of a loss function is a binary loss function, where a Sigmoid layer and the BCELoss is combined in one single class, described in pytorch.org/docs/stable/generated/torch.nn.BCEWithLogitsLoss.html.

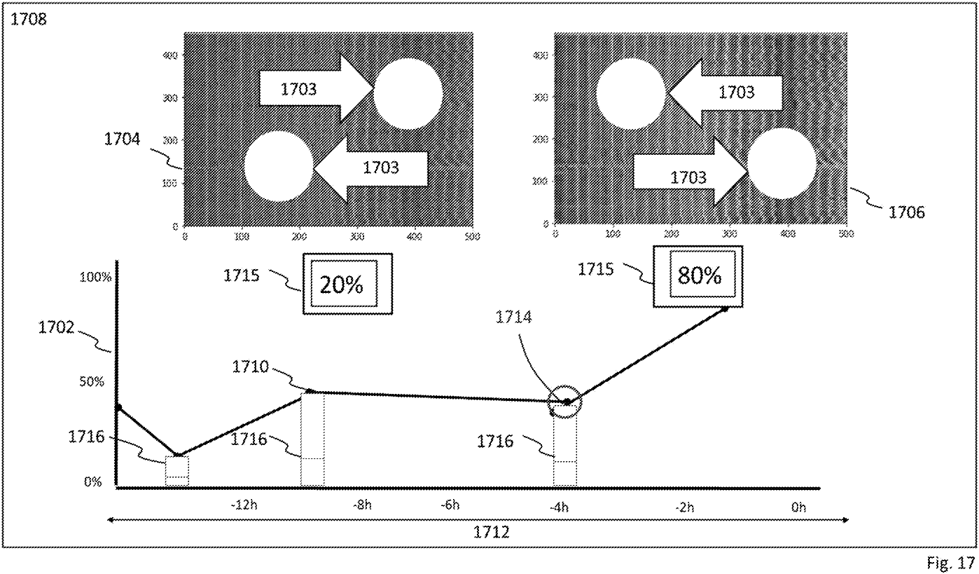

FIG. 17 illustrates an example of explanatory information for an output of a computerized model. The explanatory information provides that a user may evaluate functioning of the computerized model for detecting a threatening condition. The output 1702 of the computerized model may be provided e.g. at step 612 in FIG. 6. The explanatory information 1703 may be provided on the basis of 2D PSDs 1704, 1706 generated on the basis of the time-domain sample sequences of measurements of at least two biosignals from a subject in accordance with step 602 in FIG. 6. The explanatory information may be displayed on a user interface 1708 of a display device. The output of the computerized model based on the 2D PSDs may be obtained in accordance with step 612 of FIG. 6. The output may comprise a risk for a life-threatening condition or at least information indicating a risk for a life-threatening condition. The explanatory information 1703 may provide a mapping between the 2D PSDs 1704, 1706 input to the computerized model and the output of the computerized model 1702 generated by the computerized model in response to the input 2D PSDs.

In an example, the output of the computerized model comprises values 1710 for a risk of a life-threatening condition over a time interval 1712. Any output value 1714 generated by the computerized model can be selected to provide detailed explanatory information. Explanation can be generated from the 2D PSDs 1704, 1706 that have been input to the computerized model and explanatory information 1703 can be overlayed on the representation of the 2D PSDs. In an example the explanatory information 1703 comprises one or more portions of one or more 2D PSDs 1704, 1706 that have contributed to the selected value 1714. The 2D PSDs provide that the user may evaluate the 2D PSDs that have produced the selected output value 1714. The 2D PSDs 1704, 1706 may be divided into one or more portions and a contribution of each portion of the 2D PSDs to the risk may be evaluated. The evaluation may give a weight for each of the portions. Then, a given number, e.g. one, two, three or more of the portions that have the highest weight may be highlighted for providing the explanatory information. In this way the user may be displayed relevant portions of the 2D PSDs for verification of the life-threatening condition and assessment for the need of next actions. The weight may also present the contribution 1715 of each 2D PSD to the selected total risk or present the contribution as weight history on a stacked bar graph 1716 in order to identify the malfunctioning organ system in more detail. It should be noted that alternatively or additionally, to the 2D PSDs, raw time domain sample sequences or windowed time domain sample sequences used to generate the input data to the computerized model may be identified and serve for the explanatory information 1703 displayed to the user.

In an example, the 2D PSDs 1704, 1706 may comprise a PPG PSD and ECG PSD. The weight history provides the user to observe how a contribution 1715 of each 2D PSD has evolved from one or more past time instants to the current time instant. In this way, the user may determine whether monitoring of the life-threatening condition should be continued by a detection device and for how long the monitoring shall be continued. The contribution of each 2D PSD may vary. For example, if a contribution of one 2D PSD is higher than a contribution of another PSD, for a given length of weight history and the output value indicates an increased risk for a life-threatening condition, the user may determine that the life-threatening condition may be reliably detected by the detection device. On the other hand if based on the weight history it cannot be ascertained which of the 2D PSDs has the highest contribution because the 2D PSD having the highest contribution varies in time, the user may determine that the life-threatening condition may not be reliably detected by the detection device. If the life-threatening condition cannot be reliably detected, the user may use another method or make further examination on the subject. However, if the life-threatening condition can be reliably detected, the detection of the life-threatening condition may be continued by the detection device. Additionally the user can decide further action or change in care protocol based on the contribution of different organ systems to the risk of life-threatening condition in question.

In accordance with at least some embodiments, there is provided a method at one or more detection devices. The method comprises determining, by the one or more detection devices device, current contributions of the generated 2D PSDs 1704, 1706 to the increased risk for life-threatening condition; determining, by the one or more detection devices, a contribution history 1716 of the generated 2D PSDs to the increased risk for a life-threatening condition; displaying, by the one or more detection devices, the current contributions and the contribution history on a user interface 1708. In an example there is provided a computer program product comprising instructions which, when the program is executed by a computer, cause the computer to carry out a method in accordance with any of the embodiments described herein.

FIG. 18 illustrates an example of training a computerized model in accordance with at least some embodiments. The method may be performed by one or more data processing devices described with FIG. 1, FIG. 2 or FIG. 4 serving as a training device. Phase 1802 comprises setting one or more CM hyperparameters. Non-limiting examples of the hyperparameters comprise a temporal window size/a number of samples per window, FFT oversampling factor, positive class labeling instructions, such as temporal window start and stop values related to a life-threatening condition timestamp, 2D PSD height and width, decision thresholds and CNN hyperparameters. Examples of the CNN hyperparameters comprise neural template structure type, input channels, number of neural network layers, number of neurons in each neural network layer, number of convolution layers, number of fully connected layers, number of hidden layers, activation functions, strides, kernel sizes, dropout and/or batch normalization usage, transformer hyperparameters, such as, input channels, input axis, number of frequency bands, base frequency, maximum frequency, depth of network, number of latents, latent dimension, number of heads for cross attention, number of heads for latent self attention, dimensionality of cross attention head, dimensionality of latent self attention head, attention dropout, Fourier encoding of input data, number of self attention blocks per cross attention, whether to weight tie layers, whether to mean pool and project embeddings to number of classes at the end of the network, number of sequential 2D PSDs, and feed forward dropout, training hyperparameters, such as, regularization function, loss function, optimization function and its parameters, number of classes, number of training epochs and loss function weight coefficient(s).

Phase 1804 comprises training the CM using an evaluation data set. In an example the evaluation data set may be split into two portions, stratified by patient identifiers so that data from one patient is always present in only one portion, where the first portion is used for training the CM and the second portion is used for evaluation of the trained CM. Additional demographic and endpoint data can also be used for stratification if required for balancing the training and testing data sets for a specific life-threatening condition. During the training, a balancing sampler can be used to mitigate a class imbalance between positive and negative labels of the 2D PSDs.

Phase 1806 comprises evaluating the trained CM. The evaluation may be performed using the evaluation data set. During the training, an intermediate 2D PSD cache may be used to speed up the training, so that the computationally heavy FFT-transformation is not performed during each training epoch. If the evaluation data set has been split into portions, the portion that was not used in phase 1804 for training may be used in phase 1806 for evaluating the trained CM in order to quantify CM performance on a representative out-of-sample patient population. Another way to quantify the performance of the CM on a representative out-of-sample patient population is to use stratified cross validation where the cross validation folds are stratified by patient identifier. The evaluation may comprise calculating one or more performance metrics based on output of the trained CM to data of the evaluation data set. Example of a performance metric is the area under the precision-recall curve (AUC-PR) combining a recall (also known as sensitivity) and a precision (also called positive predictive value). Another example of a performance metric is the area under the receiver operating characteristic (AUROC) or, in the case of evaluating CM multi-class output, F1 score. Performance evaluation of the CM can be done by comparing the CM outputs directly to the true labels of the labeled 2D PSDs or population-wise where the performance of the CM is evaluated against detecting the condition on a patient regardless of the amount of labeled 2D PSDs recorded for a single patient. The population-wise performance is evaluated as taking the highest predicted output probability of any 2D PSD recorded for a patient and comparing the highest probability value to a true patient label. True patient label being true for a patient who has a life-threatening event detected at any point during the recording and false for patient who lacks any label associated with the life-threatening event in question. Population-wise performance can also be weighted with a temporal component which describes how early or late the CM detected the life-threatening condition in relation to the life-threatening event timestamp to encourage early detection of a specific life-threatening condition. If the performance metric is not satisfactory the method proceeds to phase 1802 after permuting the hyperparameters according to defined permutation function, such as a list of tested hyperparameters and selecting the next combination with, for example, a grid search algorithm. The hyperparameter permutation may also evaluate a finite combination of the different parameters and then finally select the best combination according to the defined performance metric and then proceed to phase 1808. The hyperparameter permutation may also proceed to phase 1808 when the defined performance metric exceeds a predefined threshold. Accordingly, the method proceeds to phase 1808, when the training of the CM is determined sufficient based on the performance metric.

It should be noted that more than one performance metrics may be used in phase 1806, when the CM is trained for detecting more than one life-threatening condition.

Phase 1808 comprises training the CM using a production data set with optimal hyperparameters discovered in the phase 1806. The production data set is different from the evaluation data set used in phases 1804 and 1806 when optimizing hyperparameters of the CM, but it is similarly divided into two separate training and evaluation portions stratified by patient identifiers so that data from one patient is always present in only one portion. Additional demographic and endpoint data can also be used for stratification if required for balancing the training and testing data sets for a specific life-threatening condition. During the training, a balancing sampler can be used to mitigate a class imbalance between positive and negative labels of the 2D PSDs. Production data set consists of data from representative patients that the CM would likely encounter when deployed in a production environment. The production data set may be considerably larger than the one used at the hyperparameter optimization phase.

Phase 1810 may comprise determining whether an expected performance of the CM has been achieved. The performance evaluation of the CM can be done by comparing the CM outputs directly to the true labels of the labeled 2D PSDs or population-wise where the performance of the CM is evaluated against detecting the condition on a patient regardless of the amount of labeled 2D PSDs recorded for a single patient. The population-wise performance is evaluated as taking the highest predicted output probability of any 2D PSD recorded for a patient and comparing the highest probability value to a true patient label. True patient label being true for a patient who has a life-threatening event detected at any point during the recording and false for patient who lacks any label associated with the life-threatening event in question. Population-wise performance can also be weighted with a temporal component which describes how early or late the CM detected the life-threatening condition in relation to the life-threatening event timestamp to encourage early detection of a specific life-threatening condition. If the performance metric indicates that the CM has reached at least the expected performance, defined clinically relevant, and/or similar to the performance in the hyperparameter optimization phase at 1806, the method may proceed to phase 1812 indicating that the CM is now ready for production use. If the predefined performance metric is not reached, the method proceeds to phase 1808 where more data is collected to the production data set or diagnostic procedures are performed in order to detect, for example possible overfitting to the training data.

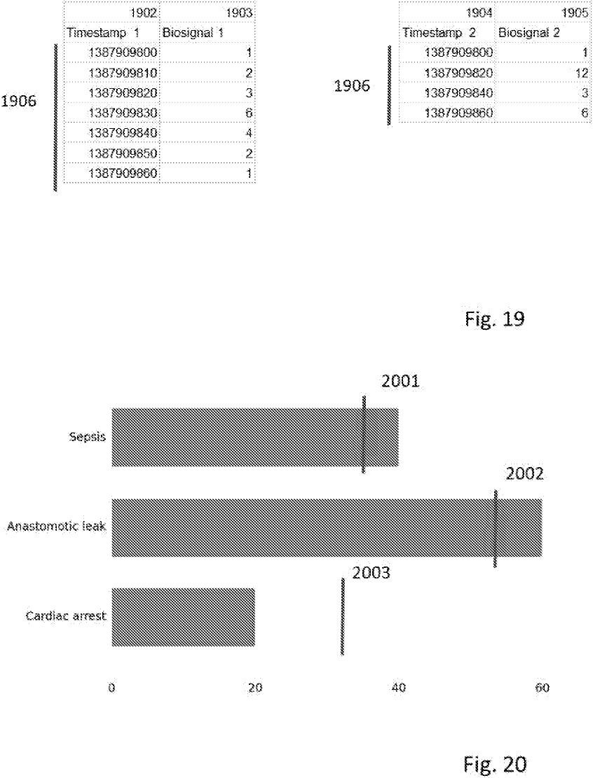

FIG. 19 illustrates time-domain sample sequences of biosignals in accordance with at least some embodiments. The sample sequences may be used for generating 2D PSDs in examples described herein. The sample sequences of biosignals are presented in columns, where the first columns 1902, 1904 includes a sample timestamp and the further columns include the samples of each biosignal 1903, 1905. Example of a sample timestamp would be a UNIX timestamp with a millisecond or a nanosecond resolution. The example comprises time-domain samples of a PPG signal 1903 and an ECG signal 1905. The ECG signal in this example is a raw digitized voltage value received from a measurement device. It should be noted that similar time-domain sample sequences may be received from measurements of other biosignals. It should be noted that the amount of samples in the biosignals may differ due to different sampling rates, but that the overall time-span 1906 indicates that the biosignals present a common, substantially overlapping segment in time. The sample sequences may be windowed based on a number of samples that may differ between different biosignals, or the sample sequences can be windowed based on a temporal window, so that each window presents a fixed segment in time for each biosignal, such as 5 minute window, 20 minute window or 12 hour window. FIG. 20 illustrates an example of information indicating at least two life-threatening conditions in accordance with at least some embodiments. The information may be displayed on a user interface of a display device. The example is related to a situation where the CM is configured to function as an ensemble, e.g. an ensemble of transformer networks or CNNs, and provides predictions for a temporally aligned set of 2D PSDs indicating the risk of multiple concurrent life-threatening conditions that are not mutually exclusive. Examples of the life-threatening conditions that are not mutually exclusive are sepsis, anastomotic leak and cardiac arrest. The bar graphs display the predictions for a single 2D PSD set that is analogous to 1602 in the context of continuous monitoring. The decision thresholds for individual life-threatening conditions can be selected similarly to 1603 by using an evaluation data set. The different models/networks of the ensemble may have different decision thresholds 2001, 2002, 2003. Since the bars of sepsis and anastomotic leak and exceed the decision threshold, the illustrated example indicates that the patient is suspected to have concurrently a sepsis and anastomotic leak.

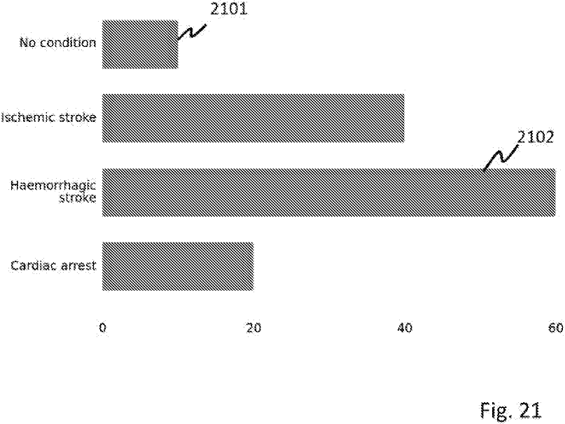

FIG. 21 illustrates an example of information indicating a differential diagnosis between multiple life-threatening conditions. The information may be displayed on a user interface of a display device. In this case the CM is configured to output a multi-class classification. The multi-class classification may comprise two or more classes which in the illustrated example comprise ‘No condition’, ‘Ischemic stroke’, ‘Haemorrhagic stroke’ and ‘Cardiac arrest’. The multi-class classification comprises scores for two or more classes the CM has been trained to detect. In the illustrated example, the scores of each class are illustrated by lengths of the bars. The indication for a life-threatening condition can be interpreted based on the class that has the highest score. In the multi-class classification case the CM may also include, in addition to one or more classes for life-threatening conditions, a special class ‘No condition’ 2101 that indicates that the patient does not have any of the life-threatening conditions recognized by the CM. Accordingly, if the output of the CM has the highest score for the ‘No condition’ -class, the patient does not have any of the life-threatening conditions recognized by the CM. In this example, the CM facilitates in making the differential diagnosis that the patient is most likely suffering from haemorrhagic stroke, given that the output score for this life-threatening condition is the highest 2102.

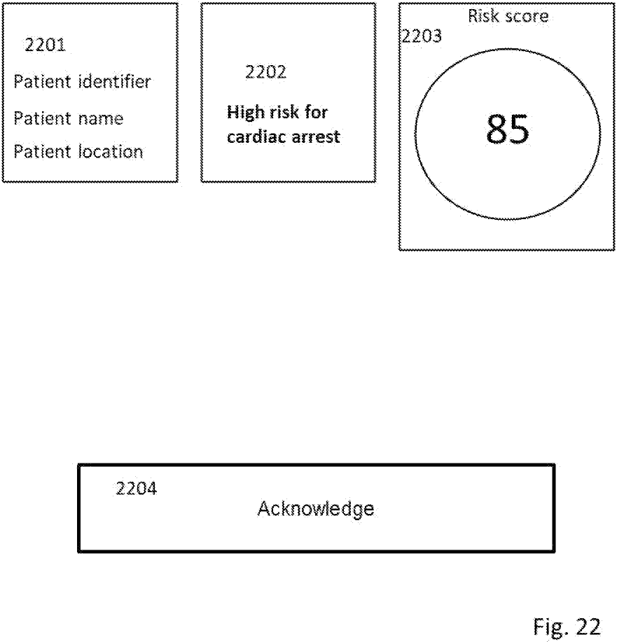

FIG. 22 illustrates an example of an alert display that may be displayed when a decision threshold for a life-threatening condition is reached for a particular patient. The information may be displayed on a user interface of a display device. The information may be accompanied with an audible alarm or action performed by the detection device via DCIF to an external system. In this non-limiting example, the alert display shows the patient demographic information and location 2201, details for the predicted life-threatening condition 2202, associated risk score 2203 and a button to acknowledge the alert 2204.

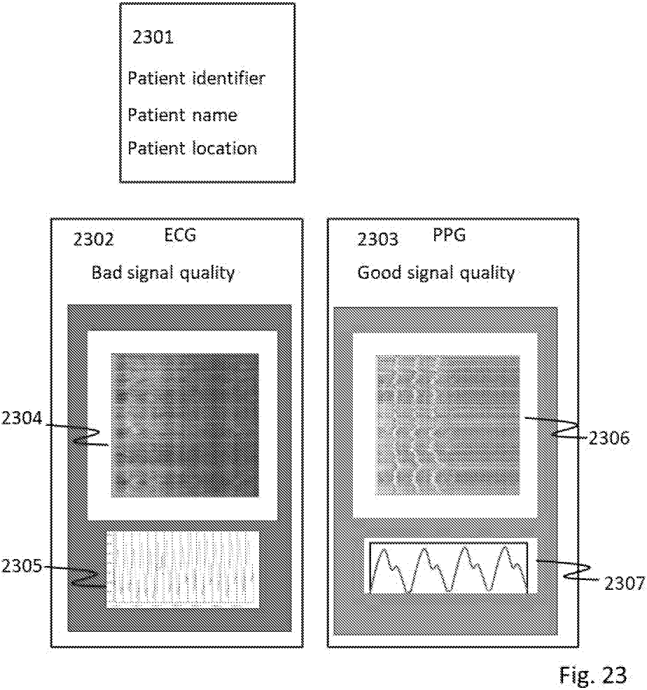

FIG. 23 illustrates an example of a quality control display for at least two recorded and/or monitored biosignals. The quality control display may be shown when the signal quality is detected to be sub-optimal for a particular patient, or the quality control display may be inspected by the care staff at any point of time. The quality control display may also be inspected in relation to any historical or current predicted output value of the CM in order to gain confidence in the predicted risk of a life-threatening condition. In the case of sub-optimal signal quality, the care staff may be instructed, for example, to reattach electrodes or perform other diagnostic procedures in order to improve signal quality to an acceptable level. The information may be displayed on a user interface of a display device. The information may be accompanied with an audible alarm or action performed by the data processing device via DCIF to an external system. In this non-limiting example, the quality control display shows the patient demographic information and location 2301, measured biosignals and automatic interpretation of their quality 2302, 2303, presentations of the 2D PSDs calculated from the biosignals 2304, 2306 and time-domain samples of the measured biosignals 2305, 2307.

The automatic interpretation of a measured biosignal quality can be performed, for example, using unsupervised learning methods, such as clustering. Accordingly, in accordance with at least some embodiments described with phases 508 and 606 in FIG. 5 and FIG. 6 quality of one or more of the biosignals may be monitored based on one or more biosignal-specific computerized models. An example would be a combination of a variational autoencoder (VAE) and an unsupervised classification algorithm, like HDBSCAN, that may be trained on a representative 2D PSD database, such as the evaluation data set. The VAE is used to project the high dimensional 2D PSD to a latent reduced dimension presentation that is still able to preserve the general high-level features of the 2D PSDs and group similar features together. This reduced dimension presentation can then be further fed to an unsupervised classifier that is able to determine how many distinct classes the 2D PSD dataset, used in the training of VAE and the unsupervised classifier, presents. Without the reduced dimension presentation, the unsupervised classifier might lack the ability to reliably classify and group the high dimensional 2D PSDs. For example, after training of the VAE, the latent bottleneck layer of the VAE can be used in conjunction with clustering and classification algorithms, like HDBSCAN or K-means clustering. The output of the clustering algorithm can be inspected and a class that mostly represents good quality 2D PSDs can be identified for each biosignal. Now, with the trained VAE, trained clustering algorithm and the knowledge of a good quality signal class, the membership and/or distance of any given 2D PSD to a centroid of a good signal quality cluster can be calculated. Relevant signal quality clusters can be visually identified by a human operator after training by sampling from the cluster population and observing the time-domain samples or 2D PSDs of the cluster members. After identification, the cluster to signal quality mapping and distance thresholds can be saved to the system thus enabling automatic classification of good and bad quality 2D PSDs for a given biosignal. Given any 2D PSD, the quality of the 2D PSD can be evaluated by first feeding the 2D PSD to a VAE and then feeding the output of the VAE bottleneck layer to a clustering algorithm that is also trained in conjunction with the VAE on a representative 2D PSD database. If the distance to a good signal quality cluster centroid, of the given 2D PSD after being fed to the VAE-clustering pipeline, exceeds a predefined threshold, then the 2D PSD can be said to be of bad quality. Otherwise the input 2D PSD is deemed to be of a good quality. The VAE can also be a Transformer-VAE, Convolutional-VAE, or a Perceiver-VAE.

A data processing device may be a training device or detection device that may comprise or be operatively connected to a computerized model for communications of input data to the computerized model and/or output data from the computerized model. The data processing device may comprise a computer program, instructions or code that may be executable by the data processing device, whereby execution of the computer program, instructions or code causes performance of a method in accordance with any of the embodiments described herein. The data processing device may comprise a memory for storing information such as a computerized model and a computer program, instructions or code.

A memory may be a computer readable medium that may be non-transitory. The memory may be of any type suitable to the local technical environment and may be implemented using any suitable data storage technology, such as semiconductor-based memory devices, magnetic memory devices and systems, optical memory devices and systems, fixed memory and removable memory. The data processors may be of any type suitable to the local technical environment, and may include one or more of general purpose computers, special purpose computers, microprocessors, digital signal processors (DSPs), graphic processing units (GPUs), deep learning processor (DLP), field-programmable gate arrays (FPGAs), quantum processors and processors based on multi-core processor architecture, as non-limiting examples.

Embodiments may be implemented in software, hardware, application logic or a combination of software, hardware and application logic. The software, application logic and/or hardware may reside on memory, or any computer media. In an example embodiment, the application logic, software or an instruction set is maintained on any one of various conventional computer-readable media. In the context of this document, a “memory” or “computer-readable medium” may be any media or means that can contain, store, communicate, propagate or transport the instructions for use by or in connection with an instruction execution system, apparatus, or device, such as a computer.

Reference to, where relevant, “computer-readable storage medium”, “computer program product”, “tangibly embodied computer program” etc., or a “processor” or “processing circuitry” etc. should be understood to encompass not only computers having differing architectures such as single/multi-processor architectures and sequencers/parallel architectures, but also specialized circuits such as field programmable gate arrays FPGA, application specify circuits ASIC, signal processing devices and other devices. References to computer readable program code means, computer program, computer instructions, computer code etc. should be understood to express software for a programmable processor firmware such as the programmable content of a hardware device as instructions for a processor or configured or configuration settings for a fixed function device, gate array, programmable logic device, etc.

The foregoing description has provided by way of exemplary and non-limiting examples a full and informative description of the exemplary embodiment of this invention. However, various modifications and adaptations may become apparent to those skilled in the relevant arts in view of the foregoing description, when read in conjunction with the accompanying drawings and the appended claims. However, all such and similar modifications of the teachings of this invention will still fall within the scope of this invention.

What is claimed is:

1. A method for one or more training devices for a computerized model, comprising:
receiving, by the one or more training devices, time-domain sample sequences of two or more biosignals from subjects;wherein the method comprises:
determining, by the one or more training devices, on the basis of computer-readable data from a subject database, information indicating timing of one or more life-threatening conditions of subjects;
windowing ₍₅₀₆₎, by the one or more training devices, the received time-domain sample sequences on the basis of a predefined window length;
generating, by the one or more training devices, two-dimensional power spectral densities, 2D PSDs, of the windowed time-domain sample sequences;
labeling, by the one or more training devices, the generated 2D PSDs to indicate a relationship to a life-threatening condition on the basis of the determined information indicating timing of one or more life-threatening conditions of the subjects;
training, by the one or more training devices, a computerized model for detection of a life-threatening condition on the basis of the labeled 2D PSDs.

2. The method according to claim 1, comprising:
scaling, by the one or more training devices, the 2D PSDs or the time-domain sample sequences to predefined range of values common to the 2D PSDs or the time-domain sample sequences.

3. The method according to claim 1, comprising: interpolating or decimating the 2D PSDs to a constant height and width.

4. The method according to claim 1, wherein the computer-readable data from a subject database comprises time instants of life-threatening conditions and/or elements whose association to one or more life-threatening conditions can be inferred, such as, diagnoses, treatments, procedures, SNOMED Clinical Terms and/or ICD-codes associated with timestamps.

5. The method according to claim 1, comprising:
applying log-transformations to the generated 2D PSDs and/or absolute value -transformations to the generated 2D PSDs; and
training the computerized model based on the log-transformations of the generated 2D PSDs and/or the absolute value -transformations of the generated 2D PSDs.

6. The method according to claim 1, comprisinges:
monitoring quality of one or more of the biosignals based on one or more biosignal-specific computerized models.

7. The method according to claim 1, comprising:
generating, by the one or more training devices, the 2D PSDs on the basis of Fast Fourier Transform, FFT, using an oversampling factor ≥ 1.

8. The method according to claim 7, wherein the training comprises:
generating, by the one or more training devices, one or more combined 2D PSDs on the basis of at least two Fast Fourier Transforms, FFTs.

9. The method according to claim 8, wherein the FFTs have the same oversampling factors or oversampling factors of the FFTs of the single 2D PSDs are different.

10. The method according to claim 1, wherein the training comprises:
applying, by the one or more training devices, a modification to a portion of at least one of the generated 2D PSDs;
training, by the one or more training devices, the computerized model on the basis of the generated 2D PSDs comprising at least one 2D PSD comprising the modification.

11. The method according to claim 1, wherein the training comprises:
flipping, by the one or more training devices, a temporal axis of at least one of the generated 2D PSDs;
training, by the one or more training devices, the computerized model on the basis of the generated 2D PSDs comprising the at least one 2D PSD comprising the flipped temporal axis.

12. The method according to claim 1, wherein after training of the computerized model has been completed, wherein the method comprises:
receiving, by the one or more training devices, time-domain sample sequences of two or more biosignals from a subject;
windowing, by the one or more training devices, the received time-domain sample sequences on the basis of the predefined window length;
generating, by the one or more training devices, two-dimensional power spectral densities, 2D PSDs, of the windowed time-domain sample sequences;
receiving, by the trained computerized model, the 2D PSDs;
outputting, by the trained computerized model, information indicating a life-threatening condition based on the 2D PSDs processed by the trained computerized model; and
controlling, by the one or more training devices, data communications interface operatively connected to the one or more training devices, to indicate an increased risk for a life-threatening condition on the basis of the output from the trained computerized model.

13. The method according to claim 1, wherein the computerized model is a convolutional neural network, a Bayesian convolutional neural network, an ensemble of convolutional neural networks, an ensemble of Bayesian convolutional neural networks, a transformer network, an ensemble of transformer networks, Bayesian transformer network or an ensemble of Bayesian transformer networks.

14. The method according to claim 13, wherein the computerized model is a transformer network, an ensemble of transformer networks, Bayesian transformer network or an ensemble of Bayesian transformer networks, and the method comprises:
forming input sequences of the generated 2D PSDs; and
training the computerized model by feeding the computerized model the formed sequences.

15. The method according to claim 1, wherein the time-domain sample sequences comprise at least one of electrocardiogram, ECG, signal, a thermocouple signal, electroencephalogram, EEG, signal, infrared signal, pressure signal, accelerometer signal, radar signal, ballistocardiographic signal, capnography signal, photoplethysmography signal, electrodermal activity signal, near-infrared spectroscopy signal, mid-infrared spectroscopy signal, transcutaneous bilirubin signal, impedance pneumography signal, electromyography, EMG, signal magnetoencephalography, MEG, signal, electrogastrogram, EGG, signal, electrical impedance tomography, EIT, signal and an invasive blood pressure signal.

16. The method according to claim 1, wherein the life-threatening condition comprises at least one of respiratory failure, sepsis, cardiac arrest, cardiac failure, congestive heart failure, renal failure, overhydration, pulmonary edema, hyper metabolic state, overexertion, brain injury, ischemic stroke, hemorrhagic stroke, multiorgan failure, anastomotic leak, internal bleeding, cardiac injury, intestinal obstruction, intestinal rupture, pulmonary embolus, opioid induced respiratory depression, seizure, over sedation, anaphylaxis, hypoxic brain damage, pneumonia, deep vein thrombosis, meningitis, malignant arrhythmia, hypovolemic shock, cardiogenic shock, obstructive shock, distributive shock, toxic shock, septic shock, myocardial infarction, wound infection, diabetic coma, endocarditis, myocarditis, pericarditis, intracranial hypertension, intestinal injury, liver failure, liver injury, pancreatitis, cardiovascular collapse, peritonitis, poisoning, drug reaction, aortic dissection and acute respiratory distress syndrome.

17. A method for one or more detection devices operatively connected to one or more devices for measuring biosignals and a data communications interface, comprising:
receiving, by the one or more detection devices, time-domain sample sequences of two or more biosignals from a subject;
windowing, by the one or more detection devices, the received time-domain sample sequences on the basis of a predefined window length;wherein the method comprises:
generating, by the one or more detection devices, two-dimensional power spectral densities, 2D PSDs, of the windowed time-domain sample sequences;
receiving, by a trained computerized model of the detection device or operatively connected to the detection device, the 2D PSDs, wherein the trained computerized model is trained on the basis of 2D PSDs labeled to indicate a relationship to a life-threatening condition;
outputting, by the trained computerized model, information indicating a life-threatening condition based on the 2D PSDs processed by the trained computerized model;
controlling, by the one or more detection devices, the data communications interface to indicate an increased risk for a life-threatening condition on the basis of the output from the trained computerized model.

18. The method according to claim 17, comprising:
controlling, by the one or more detection devices, a user interface operatively connected to the one or more detection devices, to display the increased risk for a life-threatening condition.

19. The method according to claim 17, comprising:
determining, by the one or more detection devices, the increased risk for a life-threatening condition on the basis of the output from the trained computerized model exceeding a predefined threshold.

20. The method according to claim 17, comprising:
determining, by the one or more detection devices, current contributions of the generated 2D PSDs to the increased risk for life-threatening condition;
determining, by the one or more detection devices, a contribution history of the generated 2D PSDs to the increased risk for a life-threatening condition;
displaying, by the one or more detection devices, the current contributions and the contribution history on a user interface.

21. The method according to claim 17, comprising:
filtering, by the one or more detection devices, the information indicating a life-threatening condition.

22. The method according to claim 17, comprising:
applying log-transformations to the generated 2D PSDs and/or absolute value -transformations to the generated 2D PSDs; and
feeding the log-transformations of the generated 2D PSDs and/or the absolute value -transformations of the generated 2D PSDs to the computerized model.

23. The method according claim 17, comprises:
monitoring quality of one or more of the biosignals based on one or more biosignal-specific computerized models.

24. The method according to claim 17, wherein the computerized model is a transformer network, an ensemble of transformer networks, Bayesian transformer network or an ensemble of Bayesian transformer networks, and the method comprises:
forming input sequences of the generated 2D PSDs; and
feeding the formed sequences to the computerized model.

25. A detection device operatively connected to one or more devices for measuring biosignals and a data communications interface, the detection device comprising at least one processor, and a memory comprising instructions which, when the instructions are executed by at least one processor, the detection device is caused to:
receiving, by the detection device, time-domain sample sequences of two or more biosignals from a subject;
windowing, by the detection device, the received time-domain sample sequences on the basis of a predefined window length;
generating, by the detection device, two-dimensional power spectral densities, 2D PSDs, of the windowed time-domain sample sequences;
receiving, by a trained computerized model of the detection device or operatively connected to the detection device, the 2D PSDs, wherein the trained computerized model is trained on the basis of 2D PSDs labeled to indicate a relationship to a life-threatening condition;
outputting, by the trained computerized model, information indicating a life-threatening condition based on the 2D PSDs processed by the trained computerized model;
controlling, by the detection device, the data communications interface to indicate an increased risk for a life-threatening condition on the basis of the output from the trained computerized model.

26. The detection device according to claim 25, wherein the detection device for measuring biosignals comprises at least one of electrocardiogram, ECG, signal measurement device, a thermocouple signal measurement device, electroencephalogram, EEG, signal measurement device, infrared signal measurement device, pressure signal measurement device, accelerometer signal measurement device, radar signal measurement device, ballistocardiographic signal measurement device, capnography signal measurement device, photoplethysmography signal measurement device, electrodermal activity signal measurement device, near-infrared spectroscopy signal measurement device, mid-infrared spectroscopy signal measurement device, transcutaneous bilirubin signal measurement device and impedance pneumography signal measurement device, electromyography, EMG, signal measurement device, invasive blood pressure measurement device, magnetoencephalography, MEG, signal measurement device, electrogastrogram, EGG, signal measurement device, electrical impedance tomography, EIT, signal measurement device an interface configured to connect to an electrocardiogram, ECG, signal measurement device, an interface configured to connect to a thermocouple signal measurement device, an interface configured to connect to electroencephalogram, EEG, signal measurement device, an interface configured to connect to a infrared signal measurement device, an interface configured to connect to a pressure signal measurement device, an interface configured to connect to a accelerometer signal measurement device, an interface configured to connect to a radar signal measurement device, an interface configured to connect to a ballistocardiographic signal measurement device, an interface configured to connect to a capnography signal measurement device, an interface configured to connect to a photoplethysmography signal measurement device, an interface configured to connect to a electrodermal activity signal measurement device, an interface configured to connect to a near-infrared spectroscopy signal measurement device, an interface configured to connect to a mid-infrared spectroscopy signal measurement device, an interface configured to connect to a transcutaneous bilirubin signal measurement device, an interface configured to connect to an impedance pneumography signal measurement device, an interface configured to connect to a electromyography, EMG, signal measurement device, an interface configured to connect to magnetoencephalography, MEG, signal measurement device, an interface configured to connect to electrogastrogram, EGG, signal measurement device, an interface configured to connect to electrical impedance tomography, EIT, signal measurement device and an interface configured to connect to a invasive blood pressure measurement device.

27. (canceled)

28. A training device comprising at least one processor, and a memory comprising instructions which, when the instructions are executed by at least one processor, the training device is caused to:
receiving by the training device, time-domain sample sequences of two or more biosignals from subjects;
determining by the training device, on the basis of computer-readable data from a subject database, information indicating timing of one or more life-threatening conditions of subjects;
windowing by the training device, the received time-domain sample sequences on the basis of a predefined window length;
generating by the training device, two-dimensional power spectral densities, 2D PSDs, of the windowed time-domain sample sequences;
labeling by the training device, the generated 2D PSDs to indicate a relationship to a life-threatening condition on the basis of the determined information indicating timing of one or more life-threatening conditions of the subjects;
training by the training device, a computerized model for detection of a life-threatening condition on the basis of the labeled 2D PSDs.
